(19) United States
(12) Patent Application Publication
(10) Pub. No.: US 2026/0078201 A1
(43) Pub. Date: Mar. 19, 2026

(54) ANTI-PTK7 ANTIBODY, AND USE THEREOF

(71) Applicant: UIF (University Industry Foundation), Yonsei University, Seoul (KR)

(72) Inventors: Seung-Taek LEE, Seoul (KR); Won-Sik SHIN, Seoul (KR); Si Won OH, Gyeonggi-do (KR); So-Young CHOI, Sejong (KR); Se-Ra LEE, Sejong (KR); Sang Gil KIM, Gyeonggi-do (KR)

(21) Appl. No.: 19/108,582

(22) PCT Filed: Sep. 6, 2023
(86) PCT No.: PCT/KR2023/013326

§ 371 Date: Mar. 4, 2025

(30) Foreign Application Priority Data
Sep. 6, 2022 (KR) 10-2022-0112912
Sep. 6, 2023 (KR) 10-2023-0118140

(51) Int. Cl. C07K 16/40; A61K 39/00; A61P 35/00

(52) U.S. Cl. CPC C07K 16/40; A61P 35/00; A61K 2039/505

(57) ABSTRACT

The present invention relates to an anti-PTK7 antibody and a use thereof. The anti-PTK7 antibody according to the present invention was found to inhibit angiogenesis, as well as the growth, migration, and invasion of human umbilical vein endothelial cells (HUVECs). Additionally, it effectively suppresses tumor growth in vivo, making it a promising therapeutic agent for angiogenesis-related diseases and PTK7-positive cancers. The antibody also has potential for further development as a targeted therapy for intractable cancers, positioning it as a key global therapeutic agent. Furthermore, the antibody can be humanized for clinical applications and serve as a critical component in the development of novel therapeutics.

### TECHNICAL FIELD

The present invention relates to an anti-PTK7 antibody and a use thereof.

### BACKGROUND ART

A total of 58 types of receptor protein tyrosine kinases (RPTKs) are known in humans, and each RPTK is composed of an extracellular domain to which ligands bind, a transmembrane domain, and an intracellular tyrosine kinase catalytic domain. In general, when a RPTK binds to a ligand, it is dimerized, and its cytoplasmic domain is phosphorylated and activated, inducing signal transduction.

Defective RPTKs are a subgroup of RPTKs that have lost their catalytic activity due to alterations in the tyrosine kinase domain responsible for phosphorylation. Examples of defective RPTKs identified in humans include ErbB3, protein tyrosine kinase 7 (PTK7), EphA10, EphB6, and RYK. Although such defective RPTKs are in an active state, they are nevertheless implicated in physiological processes such as oncogenesis. For example, ErbB3 binds to other ErbB family members to induce oncogenic signaling. In response, a human ErbB3-neutralizing antibody (KTN3379) has been developed as a targeted anti-cancer therapeutic agent designed to overcome resistance and is currently undergoing clinical trials.

PTK7 consists of an extracellular region with seven immunoglobulin (Ig)-like loops, a transmembrane domain, and a cytosolic region containing an inactive tyrosine kinase catalytic domain. The expression of PTK7 is upregulated in various malignancies and is negatively correlated with disease-free survival and/or overall survival in patients with cancer. PTK7 potentiates oncogenic signaling by functioning as a co-receptor for active RPTKs, such as FGFR1. Further, the expression of PTK7 is upregulated in endothelial cells, especially during tube formation, and PTK7 plays an important role in angiogenesis. Increased PTK7 expression has been observed in various types of cancer, such as colorectal cancer, where it is implicated in oncogenesis and cancer metastasis. However, due to modifications in the active site of PTK7's tyrosine kinase, developing a conventional activity inhibitor is challenging, necessitating alternative strategies for targeting the function of PTK7.

Accordingly, the present inventors developed anti-PTK7 antibodies to inhibit PTK7 functions, aiming to regulate angiogenesis, oncogenesis, and cancer metastasis.

### DISCLOSURE

### Technical Problem

Given the challenges in targeting PTK7 for therapeutic applications, the present inventors conducted intensive studies to develop neutralizing antibodies that can inhibit angiogenesis and treat various carcinomas. Through these efforts, they confirmed that specifically binding of the PTK7 antibodies to the extracellular region of PTK7 effectively inhibits the activity of PTK7, leading to the suppression of cancer cell growth, migration, invasion, and angiogenesis. This discovery forms the basis of the present invention.

The present invention provides an anti-PTK7 antibody or its functional fragment thereof, which specifically binds to protein tyrosine kinase 7 (PTK7) and comprises a heavy chain variable region and a light chain variable region,
wherein the heavy chain variable region includes CDR1-VH including an amino acid sequence of SEQ ID NO: 1, 6, 11, or 16, CDR2-VH including an amino acid sequence of SEQ ID NO: 2, 7, 12, or 17, and CDR3-VH including an amino acid sequence of SEQ ID NO: 3, 8, 13, or 18, and
the light chain variable region includes CDR1-VL including an amino acid sequence of SEQ ID NO: 4, 9, 14, or 19, CDR2-VL including Trp-Ala-Ser (WAS) or Ala-Ala-Ser (AAS), and CDR3-VL including an amino acid sequence of SEQ ID NO: 5, 10, 15, or 20.

The present invention is also directed to providing a polynucleotide encoding the antibody or a functional fragment thereof.

The present invention is also directed to providing a vector including the polynucleotide.

The present invention is also directed to providing cells transfected with the vector.

The present invention is also directed to providing a method for producing an antibody or a functional fragment thereof, which specifically binds to PTK7, the method including: culturing the cells to produce a polypeptide including light and heavy chain variable regions; and
recovering the polypeptide from the cells or a culture medium in which they are cultured.

The present invention is also directed to providing an angiogenesis inhibitor including the anti-PTK7 antibody or a functional fragment thereof.

The present invention is also directed to providing a pharmaceutical composition for preventing or treating an angiogenesis-associated disease, including the angiogenesis inhibitor.

The present invention is also directed to providing an inhibitor of the growth, migration or invasion of tumor cells, including the anti-PTK7 antibody or a functional fragment thereof.

The present invention is also directed to providing a pharmaceutical composition for preventing or treating cancer, including the inhibitor of the growth, migration or invasion of tumor cells.

The present invention is also directed to providing a method for preventing or treating an angiogenesis-associated disease, the method including: administering an anti-PTK7 antibody or a functional fragment thereof to an individual in need thereof.

The present invention is also directed to providing a use of an anti-PTK7 antibody or a functional fragment thereof for preparing a medicament for preventing or treating an angiogenesis-associated disease.

The present invention is also directed to providing a method for preventing or treating cancer, the method including: administering an anti-PTK7 antibody or a functional fragment thereof to an individual in need thereof.

The present invention is also directed to providing a use of an anti-PTK7 antibody or a functional fragment thereof for preparing a medicament for preventing or treating cancer.

However, technical problems to be solved by the present invention are not limited to the aforementioned problems, and other problems that are not mentioned may be clearly understood by those skilled in the art from the following description.

### Technical Solution

One aspect of the present invention provides an anti-PTK7 antibody or a functional fragment thereof, which specifically binds to PTK7 and includes a heavy chain variable region and a light chain variable region,
wherein the heavy chain variable region includes CDR1-VH including an amino acid sequence of SEQ ID NO: 1, 6, 11, or 16, CDR2-VH including an amino acid sequence of SEQ ID NO: 2, 7, 12, or 17, and CDR3-VH including an amino acid sequence of SEQ ID NO: 3, 8, 13, or 18, and
the light chain variable region includes CDR1-VL including an amino acid sequence of SEQ ID NO: 4, 9, 14, or 19, CDR2-VL including Trp-Ala-Ser (WAS) or Ala-Ala-Ser (AAS), and CDR3-VL including an amino acid sequence of SEQ ID NO: 5, 10, 15, or 20.

As an embodiment of the present invention, for example, the antibody or a functional thereof may include a heavy chain variable region composed of an amino acid sequence of SEQ ID NO: 21 and a light chain variable region composed of an amino acid sequence of SEQ ID NO: 22.

As another embodiment of the present invention, the antibody or a functional fragment thereof may specifically bind to the extracellular region of a PTK7 protein.

As still another embodiment of the present invention, the antibody is one or more selected from the group consisting of lgG, IgA, IgM, IgE, and IgD, and the functional fragment may be one or more selected from the group consisting of diabody, Fab, F(ab′), F(ab′)2, Fv, dsFv, and scFv.

As yet another embodiment of the present invention, the antibody or a functional fragment thereof may inhibit one or more selected from the group consisting of adhesion, wound healing, chemotactic migration, and invasion.

As yet another embodiment of the present invention, the antibody or functional fragment thereof may reduce hemoglobin (Hb) levels in a tissue.

“Tissue” refers to a tissue in which blood vessels may be generated, and the antibody or functional fragment thereof may reduce angiogenesis in the tissue, thereby reducing hemoglobin in the tissue.

The tissue may be, for example, one or more selected from the group consisting of liver, pancreas, heart, blood vessels, kidneys, skin, lungs, brain, stomach, large intestine, small intestine, duodenum, rectum, ovary, mammary gland, lymph node, biliary tract, pancreatic islet, cornea, uterus, esophagus, prostate, penis, and anus.

As yet another embodiment of the present invention, the antibody or functional fragment thereof may inhibit the phosphorylation of one or more signaling molecules selected from the group consisting of kinase insert domain receptor (KDR), extracellular-signal-regulated kinase (ERK), c-Jun N-terminal kinase (JNK), focal adhesion kinase (FAK), and tyrosine kinase Src (Src).

As yet another embodiment of the present invention, the antibody or functional fragment thereof may inhibit the interaction between PTK7 and kinase insert domain receptor (KDR).

Another aspect of the present invention provides a polynucleotide encoding the antibody or functional fragment thereof.

Still another aspect of the present invention provides a vector including the polynucleotide.

Yet another aspect of the present invention provides cells transfected with the vector.

Yet another aspect of the present invention provides a method for producing an antibody or a functional fragment thereof, which specifically binds to PTK7, the method including: culturing the cells to produce a polypeptide including heavy chain and light chain variable regions; and recovering the polypeptide from the cells or a culture medium in which they are cultured.

Yet another aspect of the present invention provides an angiogenesis inhibitor including the anti-PTK7 antibody or a functional fragment thereof as an active ingredient.

Yet another aspect of the present invention provides a pharmaceutical composition for preventing or treating an angiogenesis-associated disease, including the angiogenesis inhibitor as an active ingredient.

As an embodiment of the present invention, the angiogenesis-associated disease may be one or more selected from the group consisting of cancer, endometriosis, obesity, arthritis, arteriosclerosis, hemangioma, angiofibroma, vascular malformation, vascular adhesion, scleroderma, diabetic retinopathy, macular degeneration, neovascular glaucoma, corneal diseases caused by angiogenesis, psoriasis, telangiectasia, pyogenic granuloma, seborrheic dermatitis, and Alzheimer's disease.

Yet another aspect of the present invention provides an inhibitor of the growth, migration or invasion of tumor cells, including the anti-PTK7 antibody or a functional fragment thereof as an active ingredient.

Yet another aspect of the present invention provides a pharmaceutical composition for preventing or treating cancer, including the inhibitor of the growth, migration or invasion of tumor cells.

As an embodiment of the present invention, the cancer may be one or more selected from the group consisting of glioblastoma, brain cancer, head and neck cancer, breast cancer, lung cancer, esophageal cancer, gastric cancer, duodenal cancer, appendiceal cancer, colorectal cancer, rectal cancer, liver cancer, pancreatic cancer, gallbladder cancer, anal cancer, kidney cancer, ureteral cancer, bladder cancer, prostate cancer, penile cancer, testicular cancer, uterine cancer, ovarian cancer, vulvar cancer, vaginal cancer, and skin cancer.

Yet another aspect of the present invention provides a method for preventing or treating an angiogenesis-associated disease, the method including: administering an anti-PTK7 antibody or a functional fragment thereof to an individual in need thereof.

Yet another aspect of the present invention provides a use of an anti-PTK7 antibody or a functional fragment thereof for preparing a medicament for preventing or treating an angiogenesis-associated disease.

Yet another aspect of the present invention provides a method for preventing or treating cancer, the method including: administering an anti-PTK7 antibody or a functional fragment thereof to an individual in need thereof.

Yet another aspect of the present invention provides a use of an anti-PTK7 antibody or a functional fragment thereof for preparing a medicament for preventing or treating cancer.

### Advantageous Effects

The anti-PTK7 antibody according to the present invention was found to exhibit the effect of inhibiting angiogenesis and suppressing the growth, migration, and invasion of human umbilical vein endothelial cells (HUVECs). Consequently, it can thus be used as a therapeutic agent for angiogenesis diseases and be applied to various types of PTK7-positive cancers. Additionally, it holds promise for further development as a targeted therapeutic agent for intractable cancers, positioning it as a key global therapeutic agent in this field. Furthermore, the antibody can be converted into a humanized antibody and used as an essential component in the development of novel therapeutic drugs. It can be administrated as a stand-alone treatment or in combination with established anticancer drugs to enhance the effect of cancer therapy, maximizing therapeutic benefits.

### BRIEF DESCRIPTION OF DRAWINGS

FIG. 1 is a set of views illustrating the results of analyzing the PTK7-binding domains of anti-PTK7 mAbs, FIG. 1A is a view illustrating PTK7 and deletion mutants thereof, and FIG. 1B is a view illustrating the results of pull-down analysis to determine the PTK7-binding domains of mAb-32, mAb-43, mAb-50, and mAb-52 (SP; signal peptide, Ext; extracellular region including seven Ig domains, TM; transmembrane domain; and Cyt; cytosolic region containing a catalytically defective tyrosine kinase catalytic domain (indicated as defective TK) and His tag (composed of six histidines and designated as H6)).

FIG. 2 is a view illustrating amino acid sequence information for the entire heavy and light chain variable regions of PTK7 neutralizing monoclonal antibodies (the sequences of mAb-32 and mAb-50 are very similar, the sequences of mAb-43 and mAb-52 are very similar, mAb-32 and mAb-50 differ in a total of 9 amino acids in the CDR regions, of which 6 (blue) amino acids differ in the CDR variable region, and mAb-43 and mAb-52 differ in a total of 11 amino acids in the CDR region, of which 5 (red) amino acids differ in the CDR variable region).

FIG. 3 is a view illustrating the results of confirming the effect of anti-PTK7 mAbs on the adhesion of human umbilical vein endothelial cells (HUVECs) (*p<0.05, ** p<0.01, and ***p<0.001 vs. VEGF-alone control. +p<0.05 and ++p<0.01 vs. mAb-32-treated group).

FIG. 4 is a view illustrating the results of confirming the effect of anti-PTK7 mAbs on wound healing in the HUVEC monolayer (**p<0.01 and ***p<0.001 vs. VEGF-alone control).

FIG. 5 is a view illustrating the results of confirming the effect of anti-PTK7 mAbs on the chemotactic migration of HUVECs (*p<0.001 vs. VEGF-alone control).

FIG. 6 is a view illustrating the results of confirming the effect of anti-PTK7 mAbs on the chemotactic invasion of HUVECs (**p<0.01 and ***p<0.001 vs. VEGF-alone control).

FIG. 7 is a view illustrating the results of confirming the effect of anti-PTK7 mAbs on cytotoxicity of HUVECs (***p<0.001 vs. control incubated in the medium with 1% FBS).

FIG. 8 is a view illustrating the results of confirming the effect of PTK7 mAbs on the VEGF-induced tube formation of HUVECs in vitro (*p<0.05, **p<0.01, and ***p<0.001 vs. VEGF-alone control).

FIGS. 9A and 9B are a set of views illustrating the results of confirming the effect of PTK7 mAbs on VEGF-induced angiogenesis in vitro. FIG. 9A shows the results of confirming the effect of PTK7 mAb-32 and mAb-43, and FIG. 9B shows the results of confirming the effect of PTK7 mAb-52.

FIGS. 10A and 10B are a set of views illustrating the results of confirming the effect of PTK7 mAbs on VEGF-induced angiogenesis in vivo, confirming the results of performing a Matrigel plug assay (top) and the results of measuring the hemoglobin (Hb) content in the plugs using Drabkin's Reagent Kit 525 to quantify the degree of angiogenesis (bottom) (***p<0.001 vs. VEGF-alone control). FIG. 10A shows the results of confirming the effect of PTK7 mAb-32 and mAb-43, and FIG. 10B shows the results of confirming the effect of PTK7 mAb-52.

FIG. 11 is a view illustrating the effect of PTK7 mAbs on VEGF-induced activation of KDR and its downstream signaling proteins in HUVECs.

FIG. 12 is a view illustrating the results of confirming the effect of PTK7 mAbs on PTK7-KDR interaction.

FIGS. 13A and 13B are a view illustrating the results of confirming the effect of PTK7 mAb-52 on tumor growth in vivo, FIG. 13A is a view illustrating the results of xenografting MDA-MB-231 cells, cells, into mice, and FIG. 13B is a view illustrating the results of measuring and quantifying the tumor growth curve and the size and weight of the isolated tumors after administration of PTK7 mAb-52, after xenografting KYSE-30 cells, esophageal squamous cell carcinoma (ESCC) cells, into mice.

### MODES OF THE INVENTION

The present inventors developed four human PTK7 neutralizing monoclonal antibodies to effectively inhibit the function of PTK7, for which it is difficult to develop active inhibitors due to a modification of the active site of tyrosine kinase, and confirmed their inhibitory effects on oncogenesis, metastasis, and angiogenesis, thereby completing the present invention.

Accordingly, the present invention provides an anti-PTK7 antibody or a functional fragment thereof, which specifically binds to PTK7 and includes a heavy chain variable region and a light chain variable region, wherein the heavy chain variable region includes CDR1-VH including an amino acid sequence of SEQ ID NO: 1, 6, 11, or 16, CDR2-VH including an amino acid sequence of SEQ ID NO: 2, 7, 12, or 17, and CDR3-VH including an amino acid sequence of SEQ ID NO: 3, 8, 13, or 18, and,
the light chain variable region includes CDR1-VL including an amino acid sequence of SEQ ID NO: 4, 9, 14, or 19, CDR2-VL including Trp-Ala-Ser (WAS) or Ala-Ala-Ser (AAS), and CDR3-VL including an amino acid sequence of SEQ ID NO: 5, 10, 15, or 20.

As used herein, the term “antibody” includes an immunoglobulin molecule having immunological reactivity with a certain antigen, and includes both a polyclonal antibody and a monoclonal antibody. Furthermore, the antibody includes a form produced by genetic engineering, such as a chimeric antibody (for example, a humanized murine antibody), an antibody binding to two different types of antigen (for example, a bivalent antibody), and a bispecific antibody. In the present invention, the antibody is, for example, a monoclonal antibody.

“Antibodies of the invention and anti-PTK7 antibodies” is used herein in the broadest sense, and specifically includes binding sites that specifically bind to PTK7.

The anti-PTK7 antibody or functional fragment thereof according to the present invention may specifically bind to PTK7, and in particular may specifically adhere to the extracellular domain of PTK7 with very high affinity.

The PTK7 is not particularly limited in terms of its specific biological origin, as long as it is known as PTK7 in the art, and may be derived from mammals including, for example, mouse, human, rat, chicken, dog, or monkey, and may be one derived from a human.

Typically, an antibody has a heavy chain and a light chain, each of which includes a constant region and a variable region (the sites are also known as domains). The light and heavy chain variable regions are each composed of one domain, the heavy chain variable region (VH) or the light chain variable region (VL). Light chains and heavy chains align side-by-side, with their respective variable and constant regions positioned accordingly. They are linked by one covalent disulfide bond, while the heavy chains of two molecules, each binding to a light chain, are linked by two covalent disulfide bonds, forming the whole antibody structure. The whole antibody specifically binds to an antigen through the variable regions of the heavy and light chains, and since the whole antibody consists of two pairs of heavy and light chains (HC/LC), the whole antibody of one molecule has divalent monospecificity that binds to two identical antigens through two variable regions.

The variable region, which includes a site where an antibody binds to an antigen, includes three highly variable regions which are called complementarity-determining regions (hereinafter, CDRs) and four framework regions. The CDRs mainly serve to bind to the epitope of an antigen. The CDRs of each chain are called sequentially CDR1, CDR2, and CDR3, typically starting from the N-terminus, and are also identified by the chain in which a specific CDR is located. However, not all CDR segments are directly involved in antigen binding.

In the present invention, in the light chain variable region of the anti-PTK7 antibody or functional fragment thereof, CDR2-VL may be Trp-Ala-Ser (WAS) or Ala-Ala-Ser (AAS), and for example, the Trp-Ala-Ser (WAS) or Ala-Ala-Ser (AAS) may be derived from the anti-PTK7 antibodies (#32, #43, #50 and #52) shown in Example 2.(2) of the present application.

In the present invention, for example, the antibody or functional fragment thereof may include a heavy chain variable region composed of an amino acid sequence of SEQ ID NO: 21 and a light chain variable region composed of an amino acid sequence of SEQ ID NO: 22.

In the present invention, the antibody may be one or more selected from the group consisting of lgG, IgA, IgM, IgE, and IgD, and may be, for example, lgG. The IgG form of the antibody includes all of the lgG1, lgG2, lgG3, or lgG4 subtype forms.

The functional fragment of the present invention is a fragment of the antibody that retains the antigen-specific binding ability of the whole antibody, and the fragment possesses at least 20%, 50%, 70%, 80%, preferably 90%, 95%, 96%, 97%, 98% or 99% or 100% or more of the PTK7 affinity of the parent antibody. Specifically, the fragment may be one or more selected from the group consisting of diabody, Fab, F(ab′), F(ab′)2, Fv, dsFv, and scFv, but is not limited thereto.

The antibody or fragment thereof of the present invention may include conservative amino acid substitutions (referred to as conservative variants of the antibody) that do not substantially alter its biological activity.

In the present invention, the antibody or functional fragment thereof may inhibit one or more selected from the group consisting of adhesion, wound healing, chemotactic migration, and invasion, but is not limited thereto.

In the present invention, the antibody or functional fragment thereof may reduce hemoglobin (Hb) levels in a tissue.

In the present invention, the antibody or functional fragment thereof may inhibit the phosphorylation of one or more signaling molecules selected from the group consisting of KDR, ERK, JNK, FAK, and Src, but is not limited thereto.

In the present invention, the antibody or functional fragment thereof may inhibit the interaction between PTK7 and KDR.

In an embodiment of the present invention, the anti-PTK7 antibody or functional fragment thereof may be characterized by inhibiting cancer growth.

In a specific embodiment, the present inventors prepared the anti-PTK7 antibody and confirmed the anti-cancer effect according to the inhibition of PTK7 function thereof.

In an embodiment of the present invention, to analyze the PTK7-binding domains of anti-PTK7 mAbs, a pull-down assay was performed using anti-PTK7 mAbs. As a result, it can be seen that mAb-32 and mAb-50 can recognize the PTK7-Ig6-7 domain because they bind to PTK7-Ig1-7-His but not to other deletion mutants, and that mAb-43 and mAb-52 can recognize the PTK7 Ig2 domain because they bind to PTK7-Ig1-7-His, PTK7-Ig1-5-His, PTK-7-Ig1-4-His, PTK7-Ig1-3-His, and PTK7-Ig2-4-His, but not to PTK7-Ig3-4-His (see Example 2.(1)).

In another embodiment of the present invention, the effect of anti-PTK7 mAbs on angiogenic phenotypes (including adhesion, wound healing, chemotactic migration, and invasion) in HUVECs was analyzed. As a result, it was confirmed that mAb-32, mAb-43, mAb-50, mAb-52, and sPTK7 decreased the VEGF-induced adhesion of HUVECs. In addition, it was confirmed that mAb-32, mAb-43, mAb-50, mAb-52, and sPTK7 decreased the VEGF-induced wound healing in the HUVEC monolayer.

Furthermore, it was confirmed that mAb-32, mAb-43, and mAb-52 dose-dependently suppressed the VEGF-induced chemotactic migration in HUVECs. Further, it was confirmed that mAb-32, mAb-43, and mAb-52 dose-dependently suppressed the VEGF-induced invasion of HUVECs (see Example 2.(3)).

In still another embodiment of the present invention, capillary-like tube formation assay was performed to examine the effect of anti-PTK7 mAbs on in vitro angiogenesis. It was confirmed that 10 μg/ml of mAb-32, mAb-43, or mAb-52 suppressed VEGF-induced capillary-like tube formation in vitro (see Example 2.(4)). In addition, mouse aortic ring assay was performed to examine the effect of anti-PTK7 mAbs on ex vivo angiogenesis. It was confirmed that 10 μg/ml of mAb-32, mAb-43, or mAb-52 suppressed VEGF-induced angiogenesis ex vivo (see Example 2.(4)).

In yet another embodiment of the present invention, a Matrigel plug assay was performed to examine the effect of anti-PTK7 mAbs on angiogenesis in vivo. As a result of co-treatment with 3 μg/ml of mAb-32, mAb-43 or mAb-52 and VEGF, it was confirmed that plugs with an orange or pale red color were generated, and as a result of co-treatment with 10 μg/mL of mAb-32, mAb-43 or mAb-52 and VEGF, it was confirmed that plugs with a white or yellow color were generated. Furthermore, the degree of in vivo angiogenesis was quantified by measuring the hemoglobin (Hb) content in the plugs. As a result, it was confirmed that mAb-32, mAb-43, and mAb-52 decreased the hemoglobin level increased by VEGF (see Example 2.(5)).

In yet another embodiment of the present invention, the effect of anti-PTK7 mAbs on the VEGF-induced activation of signaling proteins in HUVECs was examined. As a result, it was confirmed that mAb-32 and mAb-43 downregulated the phosphorylation of KDR, ERK, JNK, FAK, and Src (see Example 2.(6)).

In yet another embodiment of the present invention, the effect of PTK7 mAb-52 on tumor growth in vivo was confirmed. The antitumor effect of anti-PTK7 mAb-52 was analyzed after xenografting triple-negative breast cancer (TNBC) MDA-MB-231 cells or ESCC KYSE-30 cells into mice. It was confirmed that mice intraperitoneally injected with 10 mg/kg of anti-PTK7-mAb-52 six times for a three-week period reduced tumor growth and reduced the size and weight of the tumors isolated from the mice compared to control mice (see Example 2.(8)).

From the results of the above examples, it can be seen that the anti-PTK7 antibody or functional fragment thereof according to the present invention can effectively block the function of PTK7 to effectively inhibit oncogenesis, metastasis, and angiogenesis caused by the expression or activity of PTK7 in various types of cancer, thereby achieving anti-cancer effects.

Further, the present invention provides a polynucleotide encoding the antibody or a fragment thereof.

As used herein, the term “polynucleotide” may also be replaced with “oligonucleotide” or “nucleic acid,” and includes DNA molecules (for example, cDNA or genomic DNA), RNA molecules (for example, mRNA), analogs of the above DNA or RNA molecules produced using nucleotide analogs (for example, peptide nucleic acids and non-naturally occurring nucleotide analogs), and hybrids thereof. The polynucleotide may be single-stranded or double-stranded.

The polynucleotide of the present invention is not particularly limited in its sequence, as long as it encodes the antibody or a fragment thereof of the present invention.

The polynucleotide encoding the antibody or the fragment thereof of the present invention may be obtained by methods well known in the art. For example, the polynucleotide encoding the antibody or the fragment thereof of the present invention may be synthesized using oligonucleotide synthesis techniques well known in the art, for example, the polymerase chain reaction (PCR) method, and the like, based on the DNA sequence encoding a part or all of the heavy and light chains of the antibody or the corresponding amino acid sequence.

In addition, the present invention provides a vector including the polynucleotide.

As used herein, the term “vector” is used for the purpose of replicating or expressing the polynucleotide of the present invention for recombinant production of the antibody or fragment thereof of the present invention, and generally includes one or more of a signal sequence, an origin of replication, one or more marker genes, an enhancer element, a promoter, and a transcription termination sequence. A vector of the present invention may preferably be an expression vector, and more preferably may be a vector including a polynucleotide of the present invention operably linked to a regulatory sequence, for example, a promoter.

Furthermore, the present invention provides cells transfected with the vector.

The cell of the present invention is not particularly limited in its type, as long as it is a cell that can be used to express a polynucleotide encoding an antibody or a fragment thereof included in the expression vector of the present invention. Cells transfected with the expression vector according to the invention (host cells) may be prokaryotic (for example, E. coli), eukaryotic, including yeast and other fungi, plant cells (for example, tobacco or tomato plant cells), and animal cells (for example, human cells, monkey cells, hamster cells, rat cells, mouse cells, insect cells or hybridomas derived therefrom), but preferably may be cells derived from mammals including humans.

As used herein, the term “transfection” refers to the alteration of the genotype of a host cell by the introduction of an exogenous polynucleotide, and refers to the introduction of an exogenous polynucleotide into a host cell, regardless of the method used in the transfection. An exogenous polynucleotide introduced into a host cell may be integrated into and maintained within the genome of the host cell or may be maintained without being integrated, and the present invention encompasses both.

The recombinant expression vector capable of expressing the anti-PTK7 antibody or functional fragment thereof according to the present invention may be transfected by being introduced into cells for producing the antibody or fragment thereof by a method known in the art, such as transient transfection, microinjection, transduction, cell fusion, calcium phosphate precipitation, liposome-mediated transfection, DEAE dextran-mediated transfection, electroporation, gene gun, and a known method for injecting nucleic acid into cells, but the transfection method is not limited thereto.

Further, the present invention provides a method for producing an antibody or a functional fragment thereof, which specifically binds to PTK7, the method including: culturing the cells to produce a polypeptide including heavy chain and light chain variable regions; and recovering the polypeptide from the cells or a culture medium in which they are cultured.

In the cell culture, the medium composition and the culture conditions may vary depending on the types of cells, and these may be appropriately selected and adjusted by a person skilled in the art.

The antibody molecule may be accumulated in the cytoplasm of cells, secreted from the cells, or targeted to the periplasm or supernatant by appropriate signal sequences. In addition, it is preferable to refold the produced antibody molecules and have a functional conformation using methods well known to those skilled in the art. The recovery of the polypeptide may vary depending on the characteristics of the produced polypeptide and the characteristics of the cells, which may be appropriately selected and adjusted by a person skilled in the art.

Furthermore, the present invention provides a method for the specific detection of PTK7, the method including contacting the antibody or fragment thereof with a sample and detecting the antibody or fragment thereof.

A person skilled in the art may appropriately select known methods for detecting a protein using the antibody and prepare a sample to be suitable for the selected method. Further, the sample may be cells or tissues obtained by biopsy or the like collected from a subject for whom the presence or absence of cancer or cancer metastasis is to be diagnosed, as well as blood, whole blood, serum, plasma, saliva, cerebrospinal fluid, and the like. Examples of the method for detecting a protein using the antibody include, but are not limited to, western blot, immunoblot, dot blot, immunohistochemistry, enzyme-linked immunosorbent assay (ELISA), radioimmunoassay, competitive binding assay, immunoprecipitation, and the like.

For the purpose of “detecting” the antibody or fragment thereof, it may be generally labeled with a detectable moiety. For example, the antibody or fragment thereof may be labeled with a radioisotope or fluorescent label, and various enzyme-substrate labels can be used, and examples of the enzymatic label include a luciferase such as Drosophila luciferase and bacterial luciferase, luciferin, 2,3-dihydrophthalazinediones, malate dehydrogenase, urase, a peroxidase such as horseradish peroxidase (HRPO), alkaline phosphatase, β-galactosidase, glucoamylase, lysozyme, a saccharide oxidase (for example, glucose oxidase, galactose oxidase, and glucose-6-phosphate dehydrogenase), a heterocyclic oxidase (for example, uricase and xanthine oxidase), lactoperoxidase, microperoxidase, and the like. For the technique for conjugating an enzyme to an antibody, the enzyme may be directly or indirectly conjugated to the antibody using known techniques. For example, the antibody may be conjugated to biotin, and any labels belonging to the aforementioned three broad categories may be conjugated to avidin and vice versa. Biotin selectively binds to avidin, and accordingly, the label may be conjugated to the antibody in such an indirect manner.

As another aspect of the present invention, the present invention provides an angiogenesis inhibitor including the anti-PTK7 antibody or a functional fragment thereof as an active ingredient.

Furthermore, the present invention provides a pharmaceutical composition for preventing or treating an angiogenesis-associated disease, including the angiogenesis inhibitor as an active ingredient.

In the present invention, the angiogenesis-associated disease is a disease which may be induced by the continuous occurrence of abnormal or excessive angiogenesis, and may be specifically one or more selected from the group consisting of cancer, endometriosis, obesity, arthritis, arteriosclerosis, hemangioma, angiofibroma, vascular malformation, vascular adhesion, scleroderma, diabetic retinopathy, macular degeneration, neovascular glaucoma, corneal diseases caused by angiogenesis, psoriasis, telangiectasia, pyogenic granuloma, seborrheic dermatitis, and Alzheimer's disease, but is not limited thereto.

As used herein, the term “prevention” refers to all actions that suppress symptoms caused by an angiogenesis-associated disease or delay the onset of the disease by administering the pharmaceutical composition according to the present invention.

As used herein, the term “treatment” refers to all actions that ameliorate or beneficially change symptoms caused by an angiogenesis-associated disease by administering the pharmaceutical composition according to the present invention.

As another aspect of the present invention, the present invention provides an inhibitor of the growth, migration or invasion of tumor cells, including the anti-PTK7 antibody or a functional fragment thereof as an active ingredient.

Further, the present invention provides a pharmaceutical composition for preventing or treating cancer, including the inhibitor of the growth, migration or invasion of tumor cells.

In the present invention, the cancer preferably has increased expression or function of PTK7, and may be specifically one or more selected from the group consisting of glioblastoma, brain cancer, head and neck cancer, breast cancer, lung cancer, esophageal cancer, gastric cancer, duodenal cancer, appendiceal cancer, colorectal cancer, rectal cancer, liver cancer, pancreatic cancer, gallbladder cancer, anal cancer, kidney cancer, ureteral cancer, bladder cancer, prostate cancer, penile cancer, testicular cancer, uterine cancer, ovarian cancer, vulvar cancer, vaginal cancer, and skin cancer, but is not limited thereto.

As used herein, the term “prevention” refers to all actions that suppress symptoms caused by cancer or delay the onset of the cancer by administering the pharmaceutical composition according to the present invention.

As used herein, the term “treatment” refers to all actions that ameliorate or beneficially change symptoms caused by cancer by administering the pharmaceutical composition according to the present invention.

The pharmaceutical compositions according to the present invention include an angiogenesis inhibitor or an inhibitor of the growth, migration or invasion of tumor cells, including an anti-PTK7 antibody or a functional fragment thereof, as an active ingredient, and may further include a pharmaceutically acceptable carrier. The pharmaceutically acceptable carrier is typically used in formulation, and includes saline, sterile water, Ringer's solution, buffered saline, cyclodextrin, a dextrose solution, a maltodextrin solution, glycerol, ethanol, liposomes, and the like, but is not limited thereto, and may further include other typical additives such as an antioxidant and a buffer, if necessary. Further, the oral composition according to the present invention may be formulated into an injectable formulation, such as an aqueous solution, a suspension, and an emulsion, a pill, a capsule, a granule, or a tablet by additionally adding a diluent, a dispersant, a surfactant, a binder, a lubricant, and the like. With regard to suitable pharmaceutically acceptable carriers and formulations, the composition may preferably be formulated according to each ingredient by using the methods disclosed in Remington's reference. The pharmaceutical composition of the present invention is not particularly limited in formulation, but may be formulated into an injection, an inhalant, an external preparation for skin, or the like.

The pharmaceutical composition of the present invention may be orally administered or may be parenterally administered (for example, applied intravenously, subcutaneously, intraperitoneally, or locally), and the administration dose may vary depending on a patient's condition and body weight, severity of disease, drug form, and administration route and period according to the target method, but the administration dose may be properly selected by those skilled in the art.

The pharmaceutical composition of the present invention is administered in a pharmaceutically effective amount. As used herein, “pharmaceutically effective amount” refers to an amount sufficient for treating or diagnosing diseases at a reasonable benefit/risk ratio applicable to medical treatment or diagnosis, and an effective dosage level may be determined according to factors including the type of disease of patients, the severity of disease, the activity of drugs, sensitivity to drugs, administration time, administration route, excretion rate, treatment period, and simultaneously used drugs, and other factors well known in the medical field. Meanwhile, the pharmaceutical composition according to the present invention may be administered as an individual therapeutic agent or may be administered in combination with a previously known preparation for preventing or treating an angiogenesis-associated disease or cancer. When the pharmaceutical composition of the present invention is administered in combination with the previously known preparation for preventing or treating an angiogenesis-associated disease or cancer, the composition and the preparation may be administered sequentially or simultaneously, and may be administered singly or in multiple doses. It is important to administer the composition in a minimum amount that can obtain the maximum effect without any side effects, in consideration of all the aforementioned factors, and this amount may be easily determined by a person skilled in the art.

Specifically, an effective amount of the pharmaceutical composition of the present invention may vary depending on the age, sex, condition, and body weight of a patient, the absorption of the active ingredient in the body, inactivation rate and excretion rate, disease type, and the drugs used in combination, and in general, 0.001 to 150 mg, preferably 0.001 to 100 mg of the pharmaceutical composition of the present invention per 1 kg of body weight may be administered daily or every other day or may be dividedly administered once to three times a day. However, since the effective amount may be increased or decreased depending on the administration route, the severity of obesity, gender, body weight, age, and the like, the dosage is not intended to limit the scope of the present invention in any way.

As another aspect of the present invention, the present invention provides an antibody-drug conjugate in which an anti-PTK7 antibody or a functional fragment thereof; and a drug are combined with each other.

In an embodiment of the present invention, the drug may be characterized by inhibiting one or more selected from the group consisting of adhesion, wound healing, chemotactic migration, and invasion.

In an embodiment of the present invention, the drug may be characterized by decreasing hemoglobin (Hb) levels in a tissue.

In an embodiment of the present invention, the drug may be characterized by inhibiting the phosphorylation of one or more signaling molecules selected from the group consisting of KDR, ERK, JNK, FAK, and Src.

In an embodiment of the present invention, the drug may be characterized by inhibiting the interaction between PTK7 and KDR.

In an embodiment of the present invention, the drug may be characterized by inhibiting cancer growth.

In an embodiment of the present invention, the drug is not limited to the type such as a therapeutic agent or a vaccine, and may be used without any limitations.

As still another aspect of the present invention, the present invention provides a method for preventing or treating an angiogenesis-associated disease, the method including: administering an anti-PTK7 antibody or a functional fragment thereof to an individual in need thereof.

As used herein, “individual” refers to a subject in need of treatment of a disease, and more specifically, refers to a mammal such as a human or a non-human primate, a mouse, a rat, a dog, a cat, a horse, and a cow.

As yet another aspect of the present invention, the present invention provides a use of an anti-PTK7 antibody or a functional fragment thereof for preparing a medicament for preventing or treating an angiogenesis-associated disease.

As yet another aspect of the present invention, the present invention provides a method for preventing or treating cancer, the method including: administering an anti-PTK7 antibody or a functional fragment thereof to an individual in need thereof.

As yet another aspect of the present invention, the present invention provides a use of an anti-PTK7 antibody or a functional fragment thereof for preparing a medicament for preventing or treating cancer.

In addition, the present invention provides a use of the pharmaceutical composition for preventing or treating an angiogenesis-associated disease.

Furthermore, the present invention provides a use of the pharmaceutical composition for preventing or treating cancer.

Hereinafter, the present invention will be described in more detail through examples. However, these examples are provided only for exemplarily describing the present invention, and the scope of the present invention is not limited by these examples.

### EXAMPLES

### 1. Experimental Materials and Methods

### (1) Cell Culture

Human embryonic kidney 293 (HEK293) cells were obtained from Korean Cell Line Bank (Seoul, Korea) and cultured in Dulbecco's modified Eagle's medium (Hyclone, South Logan, UT, USA) supplemented with 10% bovine serum (Gibco, Grand Island, NY, USA), 100 U/mL penicillin, and 100 μg/mL streptomycin. HUVECs were purchased from Zenbio (Durham, NC, USA) and cultured in Medium 199 (Gibco) supplemented with 20% fetal bovine serum (FBS; Hyclone), 5 U/mL heparin (Sig-ma-Aldrich, St. Louis, MO, USA), and 3 ng/ml human basic fibroblast growth factor (human bFGF) (Prospec, Ness-Ziona, Israel). HUVECs were used at passages 5-10 for the experiments. All cells were cultured at 37° C., 5% CO2, and 95% air.

### (2) Antibody Preparation

Antibodies were purchased from the following vendors: Santa Cruz Biotechnology (Santa Cruz, CA, USA) for anti-phospho-ERK (sc-7383) and anti-FAK (sc-557) antibodies; Cell Signaling Technology (Beverly, MA, USA) for anti-phospho-KDR (Tyr1175; 2478S), anti-KDR (2479S), anti-phospho-Src family (Tyr416; 2101S), anti-Src (2109S), anti-phospho-JNK (Thr183/Tyr185; 4668S), and anti-JNK (9252S) antibodies; Merck Millipore (Burlington, MA, USA) for anti-phospho-FAK antibodies (Tyr396; abt135); Bioss (Boston, MA, USA) for anti-ERK2 antibodies (bms-52068R); Sigma-Aldrich for anti-FLAG-M2 antibodies (F1804); BioLegend (San Diego, CA, USA) for anti-HA antibodies (902302); Qiagen (Cambridge, MA, USA) for anti-penta-His antibodies; AbClone (Seoul, Korea) for anti-GAPDH antibodies (abc2003); KOMA Biotech (Seoul, Korea) for horseradish peroxidase-conjugated goat anti-mouse-lgG and anti-rabbit-IgG antibodies. The production of anti-PTK7 anti-serum is described in Shin, W. S.; Maeng, Y. S.; Jung, J. W.; Min, J. K.; Kwon, Y. G.; Lee, S. T. Soluble PTK7 inhibits tube formation, migration, and invasion of endothelial cells and angiogenesis. Biochem. Biophys. Res. Commun. 2008, 371, 793-798, doi: 10.1016/j.bbrc.2008.04.168.

(3) Vector Expressing sPTK7 and sPTK7 Domains

A pcDNA3-hPTK7-Ext-His vector encoding human sPTK7 (corresponding to human PTK7 Ig1-7-His) is described in Shin, W. S.; Maeng, Y. S.; Jung, J. W.; Min, J. K.; Kwon, Y. G.; Lee, S. T. Soluble PTK7 inhibits tube formation, migration, and invasion of endothelial cells and angiogenesis. Biochem. Biophys. Res. Commun. 2008, 371, 793-798, doi: 10.1016/j.bbrc.2008.04.168. pcDNA3.1-hPTK7-Ig1-5-His, pcDNA3.1-hPTK7-Ig1-4.2-His, and pcDNA3.1-hPTK7-Ig1-3-His were generated by subcloning the complementary DNA (cDNA) fragments encoding human PTK7-Ig1-5-His, PTK7-Ig1-4.2-His, and PTK7-Ig1-3-His, respectively, into pcDNA3.1. The cDNA fragments were generated using polymerase chain reaction (PCR) with the following primer pairs: Ig1-F and Ig5-His-R; Ig1-F and Ig4.2-His-R; and Ig1-F and Ig3-His-R (Table 1). Next, the cDNA fragments were digested with EcoRI and XbaI and ligated into the pcDNA3.1 vector that was digested with EcoRI and XbaI. The pcDNA3.1-hPTK7-Ig1-4-His construct encoding human PTK7-Ig1-4-His was generated using Dpnl-mediated deletion mutagenesis with the primer pair Ig1-4-His-F and Ig1-4-His-R (Table 2) and the pcDNA3.1-hPTK7-Ig1-4.2-His vector as a template. The pcDNA3.1-hPTK7-Ig2-4-His and pcDNA3.1-hPTK7-Ig3-4-His vectors encoding human PTK7-Ig2-4-His and human PTK7-Ig3-4-His were generated using Dpnl-mediated deletion mutagenesis with the primer pair Ig2-4-His-F/Ig2-4-His-R and Ig3-4-His-F/Ig3-4-His-R (Table 2) and the hPTK7-Ig1-4-His construct as a template. All constructs were sequenced to confirm the absence of PCR errors.

TABLE 1

| Primer | Nucleotide | Nucleotide |
|---|---|---|
| name* | sequence** | position*** |
| Ig1-F | 5′-TAATACGACT | 863-882 of |
| | CACTATAGGG-3′ | MN996867 |
| Ig5- | 5′- | 1729-1746 |
| His-R | GCTCTAGATCAAT | of U40271 |
| | GATGATGATGATG | |
| | ATGGCCTGTGGCT | |
| | GAACAGGG-3′ | |
| Ig4.2- | 5′- | 1470-1453 |
| His-R | GCTCTAGATCAAT | of U40271 |
| | GATGATGATGATG | |
| | ATGCTGGGTCAGG | |
| | CAATCCAA-3′ | |
| Ig3- | 5′- | 1194-1177 |
| His-R | GCTCTAGATCAAT | of U40271 |
| | GATGATGATGATG | |
| | ATGAAGGCAGGTC | |
| | ACACGCTC-3′ | |

*F: Forward primer and R: Reverse primer
**Sequences derived from pcDNA3.1(+) and human PTK7 cDNA are shown in bold underlined and bold italic format, respectively. Sequences corresponding to the XbaI restriction site (TCTAGA), stop codon (TGA), and His tag are shown in italic, bold and underlined format, respectively.
***MN996867 and U40271 represent the GenBank accession numbers for pcDNA3.1(+) and human PTK7 cDNA, respectively.

TABLE 2

| Primer | Nucleotide | Nucleotide |
|---|---|---|
| name* | sequence** | position*** |
| Ig1-4- | 5′- | 1377-1389 of |
| His-F | CATCACTGTGGCC | U40271 and |
| | CATCATCATCATC | 997-1001 of |
| | ATCATTGATCTAG | MN996867 |
| | AGGGCC-3′ | |
| Ig1-4- | 5′-GATGATGATG | 1389-1362 of |
| His-R | ATGGGCCACAGTG | U40271 |
| | ATGTTGACATCCT | |
| | GTCTC-3′ | |
| Ig2-4- | 5′-GTCTTCATCA | 259-273 and |
| His-F | AGCAGTGGATTGA | 529-553 of |
| | GGCAGGTCCTGTG | U40271 |
| | GTCC-3′ | |
| Ig2-4- | 5′-CCTGCCTCAA | 542-529 and |
| His-R | TCCACTGCTTGAT | 273-246 of |
| | GAAGACAATGGCT | U40271 |
| | GTCTGG-3′ | |
| Ig3-4- | 5′-GTCTTCATCA | 259-273 and |
| His-F | AGCAGGATGAAAG | 823-847 of |
| | CTTTGCCAGGGTG | U40271 |
| | GTGC-3′ | |
| Ig3-4- | 5′- | 837-823 and |
| His-R | GGCAAAGCTTTCA | 273-246 of |
| | TCCTGCTTGATGAA | U40271 |
| | GACAATGGCTGTCT | |
| | GG-3′ | |

*F: Forward primer and R: Reverse primer
**Sequences derived from human PTK7 cDNA and pcDNA3.1(+) are shown in bold italic and bold underlined format, respectively. Sequences corresponding to the XbaI restriction site (TCTAGA), stop codon (TGA), and His-tag are shown in italic, bold and underlined format, respectively.
***U40271 and MN996867 represent the GenBank accession numbers for human PTK7 cDNA and pcDNA3.1 (+), respectively.

(4) Expression and Purification of sPTK7 and sPTK7 Domains in HEK293 Cells

The expression vectors for His-tagged sPTK7 and sPTK7 domains were transfected into HEK293 cells using the calcium phosphate method, following a protocol described in Shin, W. S.; Shim, H. J.; Lee, Y. H.; Pyo, M.; Park, J. S.; Ahn, S. Y.; Lee, S.-T. PTK6 Localized at the Plasma Membrane Promotes Cell Proliferation and Migration Through Phosphorylation of Eps8. J. Cell. Biochem. 2017, 118, 2887-2895, doi:10.1002/jcb.25939. To select the transfected cells, the cells were cultured in the presence of 1.2 mg/mL G418 (AG Scientific, CA, USA) for 2 weeks. Stable single cell clones or mixed cell populations expressing His-tagged proteins were cultured in a medium with 0.6 mg/mL G418. Serum-free conditioned medium from cells stably expressing His-tagged proteins (for 4-5 days) was subjected to ammonium sulfate precipitation at 70% saturation. The precipitates were dissolved in phosphate-buffered saline (PBS; 137 mM NaCl, 10 mM Na₂HPO₄, 2.7 mM KCl, and 2 mM KH₂PO₄; pH 7.4) containing 1 mM phenylmethyl-sulfonyl fluoride (PMSF) and 1 mM ethylenediaminetetraacetic acid (EDTA) and dialyzed against PBS. Thereafter, His-tagged proteins were purified using Ni²+-NTA agarose (Qiagen, Hilden, Germany).

(5) Anti-PTK7 mAbs

Mouse anti-PTK7 hybridoma cell lines were established using purified human sPTK7 as the antigen (AbFrontier, Seoul, Korea). Anti-PTK7 mAbs were purified from ascites obtained via intraperitoneal injection of hybridomas into mice (AbClone).

(6) Analysis of Binding Domains of Anti-PTK7 mAbs

Purified sPTK7-His and its deletion domains were incubated with anti-PTK7 mAbs at a 1:1 molar ratio for 2 h at 4° C. and pulled down with Ni²+-NTA agarose resins (Qiagen, Cambridge, MA, USA). Protein-bound resins were washed twice with PBS containing 0.1% Tween 20. Pulled-down proteins were resuspended in sodium dodecyl sulfate (SDS) sample buffer and subjected to western blotting.

### (7) Western Blotting

Western blotting was performed as described in Choi, Y. E.; Song, M. J.; Hara, M.; Imanaka-Yoshida, K.; Lee, D. H.; Chung, J. H.; Lee, S.-T. Effects of Tenascin C on the Integrity of Extracellular Matrix and Skin Aging. Int. J. Mol. Sci. 2020, 21, doi: 10.3390/ijms21228693. Briefly, cell lysates or pull-down proteins were resuspended in SDS sample buffer and subjected to SDS-polyacrylamide gel electrophoresis (SDS-PAGE). The resolved proteins were blotted on a polyvinylidene difluoride membrane (Millipore, Bedford, MA, USA). The membrane was incubated with the indicated antibodies. Immunoreactive signals were detected using Immobilon western chemiluminescent HRP substrate (Millipore, Bedford, MA, USA) and Amersham ImageQuant 800 (Cytiva, Marlborough, MA, USA).

### (8) Adhesion Assay

HUVECs were starved for 6 h in M199 medium containing 1% FBS and the cells were resuspended in the same medium. The cell suspension (1×10⁴ cells/100 μL) was pretreated with anti-PTK7 mAbs (10 μg/mL) or human sPTK7 (4 μg/mL) for 30 min at 25° C. and loaded into gelatin-coated 96-well plates. Then, the cells were incubated with 10 ng/ml human vascular endothelial growth factor (VEGF) (KOMA Biotech) for 1 h. The stained cells were lysed with 1% SDS and the absorbance of the mixture at 600 nm was measured.

### (9) Wound Healing Assay

The monolayer of HUVECs grown in 12-well plates was starved in the M199 medium containing 1% FBS for 6 h, and a scratch was introduced into the monolayer using a micropipette tip. The cells were washed to remove debris and pre-treated with anti-PTK7 mAbs (10 μg/mL) or human sPTK7 (4 μg/mL) in M199 medium with 1% FBS. Next, the cells were incubated with 10 ng/ml human VEGF for 14 h and observed under a light microscope.

### (10) Chemotactic Migration and Invasion Assay

HUVECs were starved in the M199 medium containing 1% FBS for 6 h. Chemotactic migration and invasion assays were performed with minor modifications in Shin, W. S.; Maeng, Y. S.; Jung, J. W.; Min, J. K.; Kwon, Y. G.; Lee, S. T. Soluble PTK7 inhibits tube formation, migration, and invasion of endothelial cells and angiogenesis. Biochem. Biophys. Res. Commun. 2008, 371, 793-798, doi: 10.1016/j.bbrc.2008.04.168. HUVECs were pre-treated with anti-PTK7 mAbs (3 and 10 μg/mL) or human sPTK7 (4 μg/mL) for 30 min at 25° C. prior to loading the cells into the upper compartment of Transwell. Cells that migrated to the bottom surface of the filter were fixed with 3.7% paraformaldehyde in PBS, stained with 0.02% crystal violet, and analyzed under a light microscope (Olympus, Tokyo, Japan). The stained cells were lysed with 1% SDS and the absorbance at 600 nm of the mixture was measured.

### (11) Capillary-Like Tube Formation Assay

Capillary-like tube formation assays were performed as described in Lee, Y. H.; Park, J. H.; Cheon, D. H.; Kim, T.; Park, Y. E.; Oh, E. S.; Lee, J. E.; Lee, S.-T. Processing of syndecan-2 by matrix metalloproteinase-14 and effect of its cleavage on VEGF-induced tube formation of HUVECs. Biochem. J. 2017, 474, 3719-3732, doi: 10.1042/bcj20170340. Briefly, HUVECs were starved in M199 medium containing 1% FBS for 6 h, harvested with trypsin, and resuspended in the same medium. Cells (2×10⁵ cells/well of a 24-well plate) were pre-treated with anti-PTK7 mAbs (10 μg/mL) or sPTK7 (4 μg/mL) for 30 min and loaded on a 24-well plate whose wells were pre-coated with 300 μL of growth-factor-reduced Matrigel (Corning, Bedford, MA, USA). The cells were then incubated with a final 20 ng/ml human VEGF at 37° C. for 16 h and analyzed using light microscopy. The number of capillary-like tubes was quantified using ImageJ angiogenesis analyzer.

### (12) Mouse Aortic Ring Assay

The aortic ring assay was performed as described in Bellacen, K.; Lewis, E. C. Aortic ring assay. J. Vis. Exp. 2009, doi: 10.3791/1564. Briefly, the thoracic aorta of mice (aged 6-7 weeks) was transferred to a Petri dish filled with cold PBS, and the surrounding fat tissue was removed. The aorta was sliced using a surgical blade and placed in the center of solidified growth-factor-reduced Matrigel (150 μL). The samples were incubated for 20 min at 37° C. in a 48-well dish. Additional Matrigel (150 μL) was added to the top of each ring, and the samples were incubated at 37° C. for 20 min. M199 medium with 1% FBS (200 μL) containing 20 ng/ml mouse VEGF (KOMA Biotech) with or without anti-PTK7 mAbs (10 μg/mL) or sPTK7 (4 μg/mL) was added to each well. The medium was replaced once every 5 days. After 12 days, the outgrowth of cells from the aorta was analyzed under a phase-contrast microscope.

### (13) Matrigel Plug Assay

The Matrigel plug assay was performed as described in Shin, W. S.; Na, H. W.; Lee, S.-T. Biphasic effect of PTK7 on KDR activity in endothelial cells and angiogenesis. Biochim. Biophys. Acta 2015, 1853, 2251-2260, doi: 10.1016/j.bbamcr.2015.05.015. Briefly, growth-factor-reduced Matrigel (0.5 mL) containing 32 U heparin and 250 ng of mouse VEGF or mouse VEGF plus anti-PTK7 mAbs (3 and 10 μg/mL) was subcutaneously injected into female C57BL/6 mice aged 4 weeks. After 12 days, the mice were sacrificed and the plugs were retrieved. The hemoglobin (Hb) content in the plugs was measured using Drabkin's reagent kit 525 (Sigma-Aldrich) to quantify blood vessel formation.

### (14) Analysis of Signaling Proteins in HUVECs

Subconfluent HUVECs were starved in the M199 medium supplemented with 1% FBS for 6 h. The cells were pre-incubated with anti-PTK7 mAbs (10 μg/mL) or sPTK7 (4 μg/mL) for 30 min. Next, the cells were stimulated with 10 ng/ml human VEGF for 2 min to analyze receptor phosphorylation, 1 h to analyze the phosphorylation of FAK, or 10 min to evaluate the phosphorylation of other signaling molecules. The cells were then lysed with radioimmunoprecipitation assay lysis buffer containing 1 mM Na3VO4 and 5 mM NaF.

### (15) Co-Expression of PTK7 and KDR in HEK293 Cells

The lentiviral transfer vector pHRST-hPTK7-FLAG-IRES-eGFP encoding human PTK7 with a C-terminal FLAG tag is described in Shin, W. S.; Park, M. K.; Kim, J. H.; Oh, S. W.; Jang, J. Y.; Lee, H.; Lee, S.-T. PTK7, a Catalytically Inactive Receptor Tyrosine Kinase, Increases Oncogenic Phenotypes in Xenograft Tumors of Esophageal Squamous Cell Carcinoma KYSE-30 Cells. Int. J. Mol. Sci. 2022, 23, doi: 10.3390/ijms23042391. Lentiviruses expressing PTK7-FLAG were propagated in HEK293T cells by co-transfection of pHRST-hPTK7-FLAG-IRES-eGFP, a packaging vector psPAX2, and an envelope vector pMD2.G (Addgene, Cambridge, MA, USA). Subconfluent HEK293 cells were infected with PTK7-FLAG lentiviruses. HEK293 cells expressing PTK7-FLAG were transfected with pcDNA3.1-Kozak-KDR encoding human KDR with a C-terminal HA tag using the calcium phosphate method.

### (16) Pull-Down Assay

Subconfluent HEK293 cells co-expressing PTK7-FLAG and KDR-HA were incubated with anti-PTK7 mAbs (10 μg/mL) or sPTK7 (4 μg/mL) for 2 h. Cells were lysed with NP-40 lysis buffer (50 mM Tris-HCl (pH 7.4), 150 mM NaCl, and 1% NP-40) containing 5 mM NaF, 1 mM NasVO4, and protease inhibitor cocktail III (Calbiochem, La Jolla, CA, USA). The lysates were incubated with mouse anti-FLAG M2 antibodies (Sigma-Aldrich) for 2 h. The protein-bound resins were then washed with NP-40 lysis buffer. Pulled-down proteins were resuspended in SDS sample buffer and subjected to western blotting.

### (17) Xenograft Assay of Tumors in Mice

TNBC MDA-MB-231 cells (1×10⁶ cells) were resuspended in 0.2 ml of a 1:1 mixture of PBS and Matrigel, and then transplanted subcutaneously into the backs of mice. About 2 weeks after inoculation, when the tumor volume reached approximately 100 mm3, PBS or anti-PTK7 mAb-52 was administered intraperitoneally at 10 mg/kg twice a week for 3 weeks. After administration of the test substance, the growth of tumors was monitored and the size of tumors was measured over a total period of 5 weeks, and after the experiment was completed, the tumors were excised, and their sizes and weights were measured.

ESCC KYSE-30 cells (1×10⁶ cells) were resuspended in 0.2 ml of a 1:1 mixture of PBS and Matrigel, and then transplanted subcutaneously into the backs of mice. About 1 week after inoculation, when the tumor volume reached approximately 100 mm3. PBS or anti-PTK7 mAb-52 was administered intraperitoneally at 10 mg/kg twice a week for 3 weeks. After administration of the test substance, the growth of tumors was observed and the size of tumors was measured over a total period of 4 weeks, and after the experiment was completed, the tumors were excised and their sizes and weights were measured.

### (18) Statistical Analysis

The means between two groups were compared using Student's t-test. All data obtained from at least three independent experiments are expressed as mean±standard deviation. Differences were considered significant at p<0.05.

### 2. Experimental Results

(1) Analysis of the PTK7-Binding Domains of Anti-PTK7 mAbs

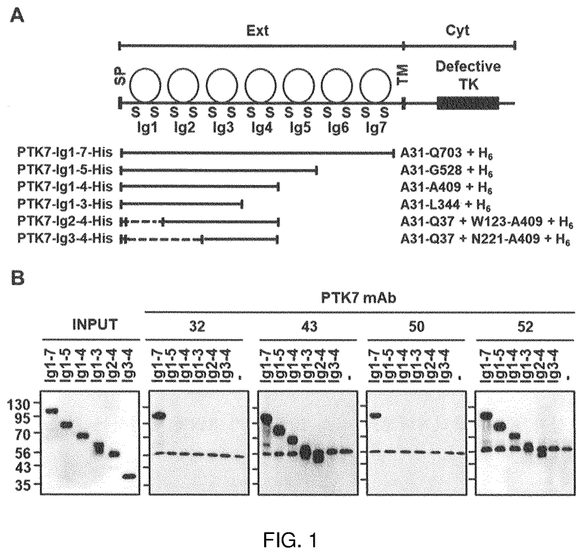

To analyze the PTK7-binding domains of anti-PTK7 mAbs, a pull-down assay was performed using anti-PTK7 mAbs. The deletion mutants of the extracellular region of PTK7 (sPTK7; named as PTK7-Ig1-7-His for comparison) were used for the analysis: PTK7-Ig1-5-His, PTK7-Ig1-4-His, PTK7-Ig1-3-His, PTK7-Ig2-4-His, and PTK7-Ig3-4-His (FIG. 1A). It can be seen that mAb-32 and mAb-50 can recognize the PTK7-Ig6-7 domain because they bind to PTK7-Ig1-7-His but not to other deletion mutants, and that mAb-43 and mAb-52 can recognize the PTK7 Ig2 domain because they bind to PTK7-Ig1-7-His, PTK7-Ig1-5-His, PTK-7-Ig1-4His, PTK7-Ig1-3-His, and PTK7-Ig2-4-His, but not to PTK7-Ig3-4-His (FIG. 1B).

(2) Sequence Analysis of the Complementary Determining Regions (CDRs) of Anti-PTK7 mAbs

The amino acid sequences of the hypervariable regions of the heavy and light chains of an antibody, that is, immunoglobulin (Ig), are called the complementarity determining regions (CDRs). The CDR sequences of an Ig provide contact residues important for antibody binding to an antigen, and there are three CDRs in each of the heavy and light chains.

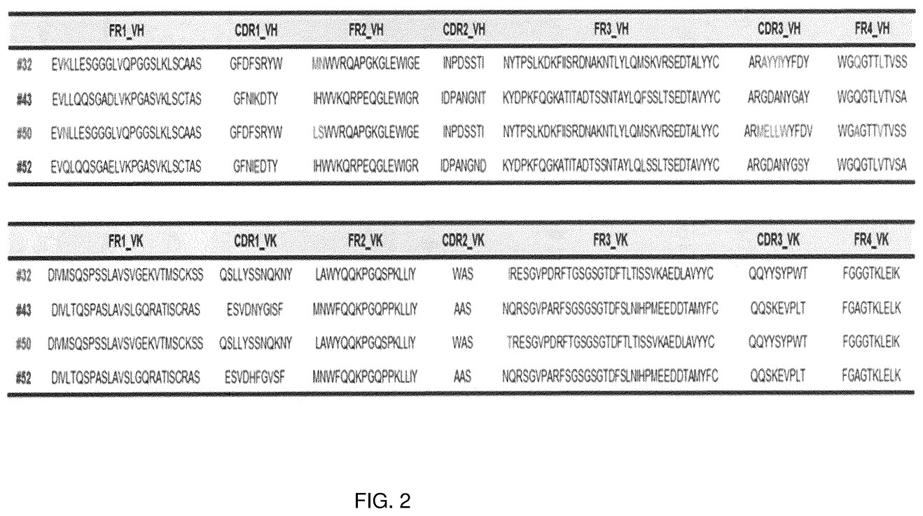

Accordingly, to examine the CDR sequences of PTK7 neutralizing monoclonal antibodies (mAb-32, mAb-43, mAb-50, and mAb-52), the present inventors isolated total RNA from hybridoma cells secreting the antibodies, synthesized cDNA using oligo-dT15 and random hexamers, amplified it with a primer set capable of amplifying the hypervariable region of Ig, cloned PCR products to confirm the sequence for each clone, and analyzed the CDR base sequences and amino acid sequences using IGBLAST Tool (https://www.ncbi.nlm.nih.gov/igblast/). The CDR amino acid sequences of the four PTK7 neutralizing monoclonal antibodies derived through the analysis are shown in the following Table 3, and the amino acid sequence information for the entire heavy chain variable region and light chain variable region is shown in FIG. 2.

TABLE 3

| | | Amino acid | SEQ |
|---|---|---|---|
| PTK7 | | sequence | ID |
| mAb | Part | (N-C) | NO: |
| #32 | CDR1_VH | GFDFSRYW | 1 |
| | CDR2_VH | INPDSSTI | 2 |
| | CDR3_VH | ARAYYIYYFDY | 3 |
| | CDR1_VL | QSLLYSSNQKNY | 4 |
| | CDR2_VL | WAS | — |
| | CDR3_VL | QQYYSYPWT | 5 |
| #43 | CDR1_VH | GFNIKDTY | 6 |
| | CDR2_VH | IDPANGNT | 7 |
| | CDR3_VH | ARGDANYGAY | 8 |
| | CDR1_VL | ESVDNYGISF | 9 |
| | CDR2_VL | AAS | — |
| | CDR3_VL | QQSKEVPLT | 10 |
| #50 | CDR1_VH | GFDFSRYW | 11 |
| | CDR2_VH | INPDSSTI | 12 |
| | CDR3_VH | ARMELLWYFDV | 13 |
| | CDR1_VL | QSLLYSSNQKNY | 14 |
| | CDR2_VL | WAS | — |
| | CDR3_VL | QQYYSYPWT | 15 |
| #52 | CDR1_VH | GFNIEDTY | 16 |
| | CDR2_VH | IDPANGND | 17 |
| | CDR3_VH | ARGDANYGSY | 18 |
| | CDR1_VL | ESVDHFGVSF | 19 |
| | CDR2_VL | AAS | — |
| | CDR3_VL | QQSKEVPLT | 20 |

(3) Effect of Anti-PTK7 mAbs on Angiogenic Phenotypes in HUVECs

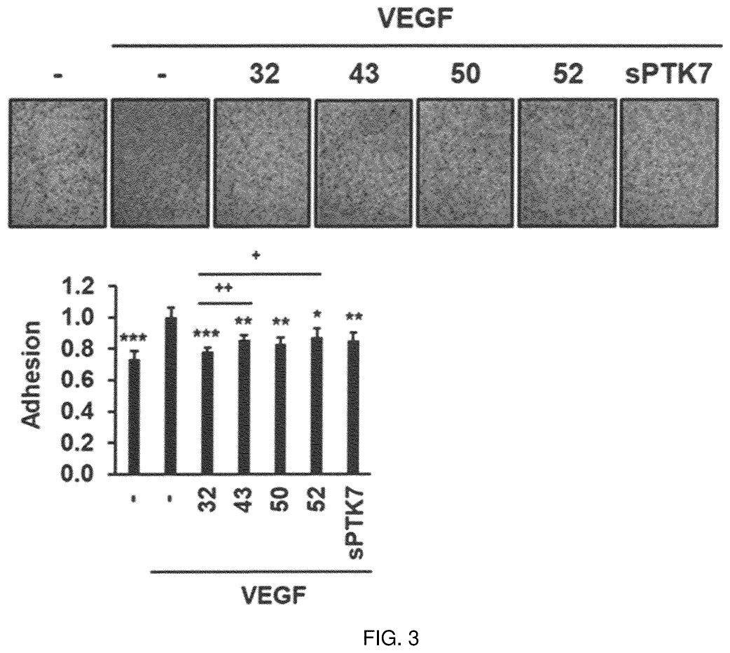

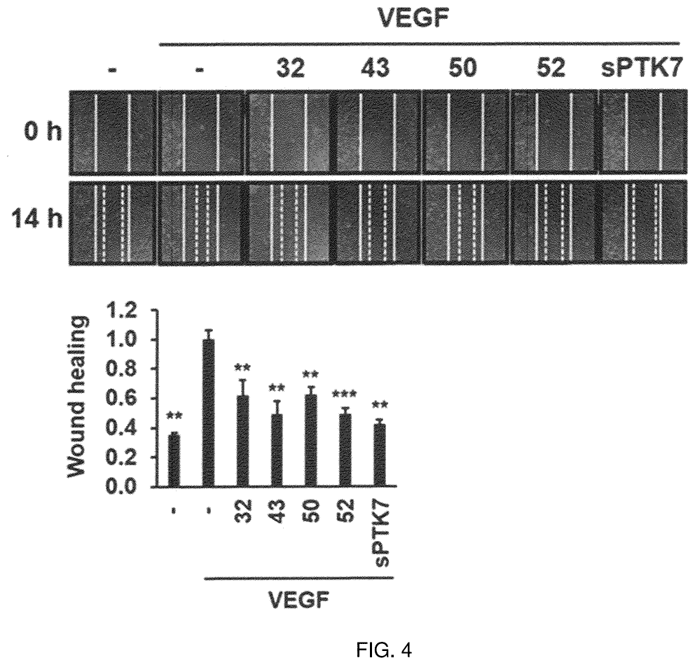

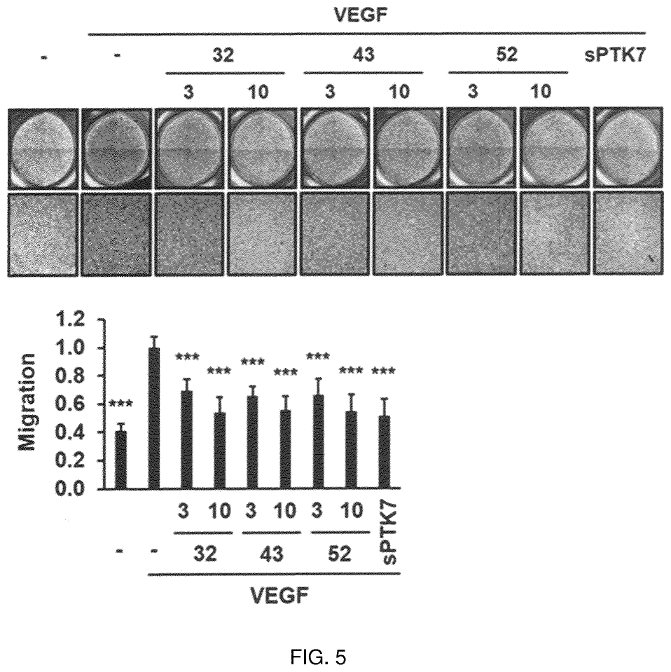

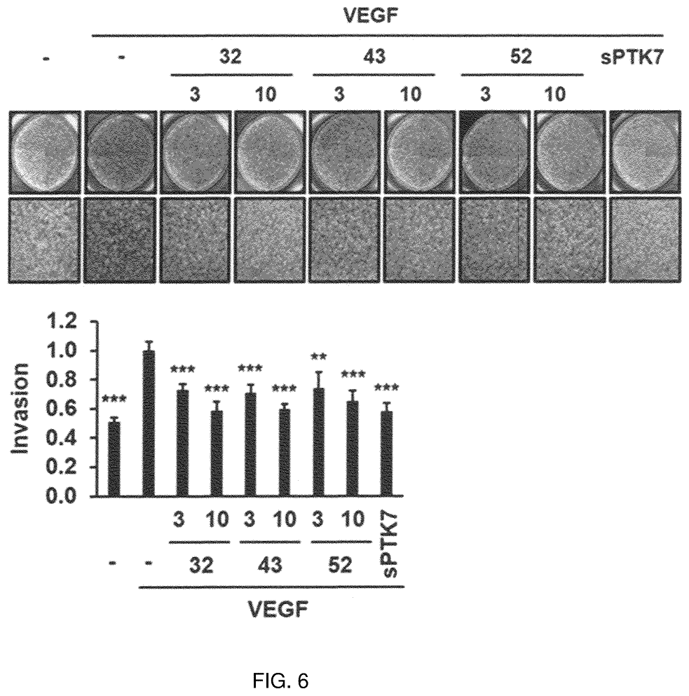

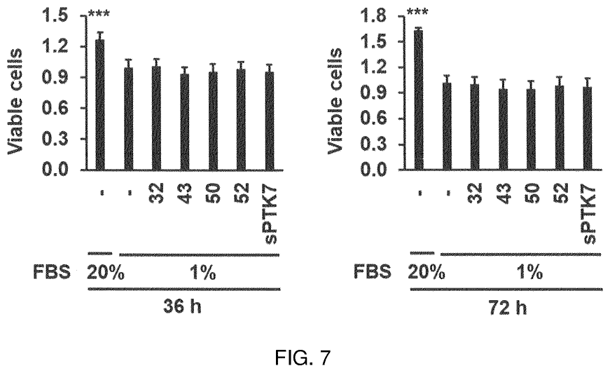

The effect of anti-PTK7 mAbs on angiogenic phenotypes (including adhesion, wound healing, chemotactic migration, and invasion) in HUVECs was analyzed. As high concentrations of sPTK7 inhibit angiogenic phenotypes, sPTK7 (4 μg/mL) was used as a positive control to inhibit PTK7 functions. As a result, it was confirmed that mAb-32, mAb-43, mAb-50, mAb-52 (10 μg/mL each), and sPTK7 decreased the VEGF-induced adhesion of HUVECs to 78.2%±2.5%, 85.5%±3.1%, 83.2%±4.0%, 87.3%±5.5%, and 85.3±5.0%, respectively (FIG. 3). In addition, it was confirmed that mAb-32, mAb-43, mAb-50, mAb-52 (10 μg/mL each), and sPTK7 decreased the VEGF-induced wound healing in the HUVEC monolayer to 62.1%±9.9%, 49.0%±8.8%, 62.2%±5.4%, 49.1%±3.8%, and 42.0%±3.0%, respectively (FIG. 4). Furthermore, it was confirmed that mAb-32, mAb-43, and mAb-52 dose-dependently suppressed the VEGF-induced chemotactic migration in HUVECs, and at a concentration of 10 μg/mL, mAb-32, mAb-43, mAb-52, and sPTK7 decreased the VEGF-induced chemotactic migration of HUVECs to 53.8%±10.1%, 55.1%±10.2%, 54.5%±11.9%, and 50.8%±12.4%, respectively (FIG. 5). Further, it was confirmed that mAb-32, mAb-43, and mAb-52 dose-dependently suppressed the VEGF-induced invasion of HUVECs, and at a concentration of 10 μg/mL, mAb-32, mAb-43, mAb-52, and sPTK7 decreased the VEGF-induced invasion of HUVECs to 58.6%±6.2%, 59.7%±3.5%, 65.2%±7.2%, and 57.8%±5.9%, respectively (FIG. 6). Although the ability of different anti-PTK7 mAbs to inhibit migration, wound healing, and invasion was not significantly different, it was confirmed that the adhesion of the mAb-32-treated group was significantly lower than that of the mAb-43-treated and mAb-52-treated groups (FIG. 3). In addition, it was confirmed that mAb-32, mAb-43, mAb-50, mAb-52 (10 μg/mL), and sPTK7 (4 μg/mL) did not exert cytotoxic effects on HUVECs (FIG. 7). Therefore, the anti-PTK7 mAbs that the present inventors tested significantly inhibited VEGF-induced angiogenic phenotypes in HUVECs.

(4) Effect of Anti-PTK7 mAbs on Angiogenesis In Vitro and Ex Vivo

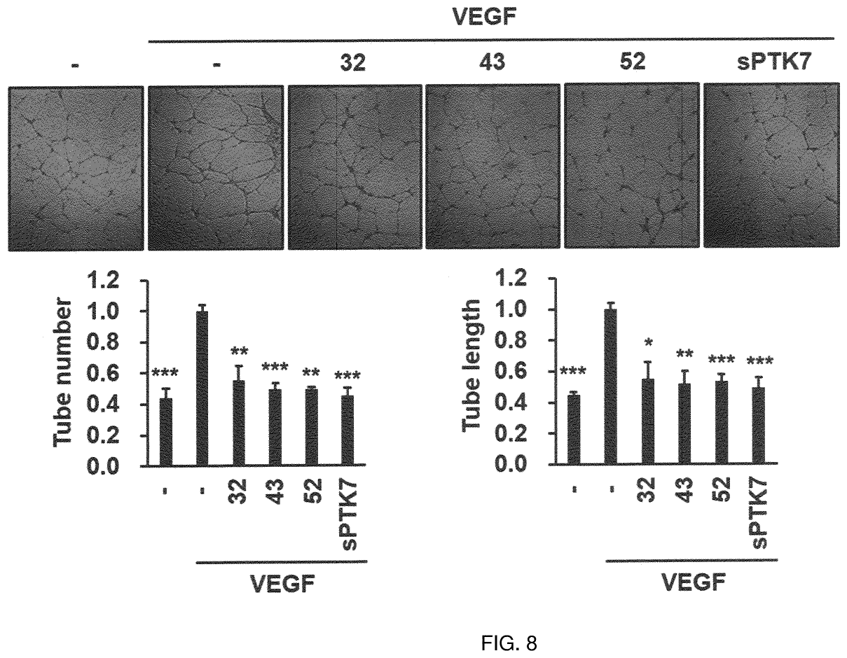

The effect of anti-PTK7 mAbs on angiogenesis in vitro was analyzed using the capillary-like tube formation assay. VEGF (20 ng/ml) induced capillary-like tube formation in HUVECs cultured on Matrigel. However, it was confirmed that mAb-32, mAb-43, mAb-52 (10 μg/mL), and sPTK7 (4 μg/mL) decreased the VEGF-induced capillary-like tube formation to 55.2%±9.3%, 49.4%±3.8%, 49.4%±1.3%, and 45.2%±5.0%, respectively (FIG. 8).

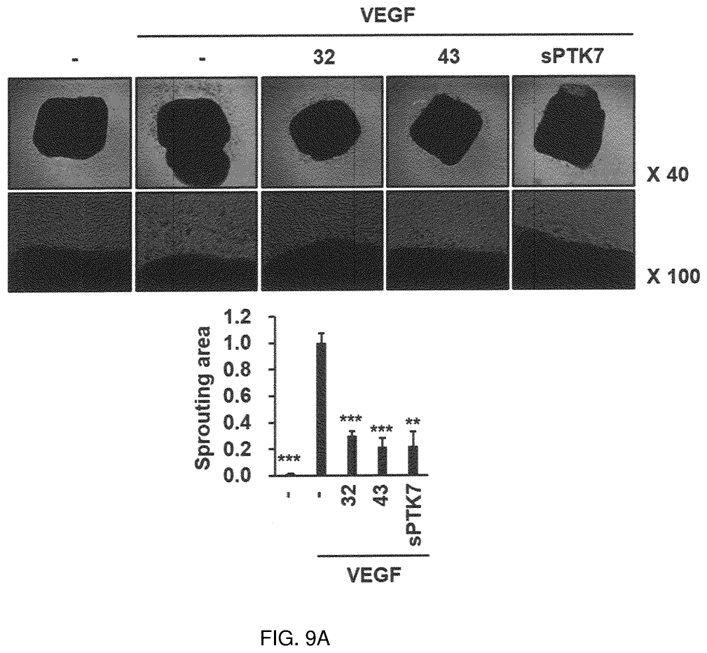

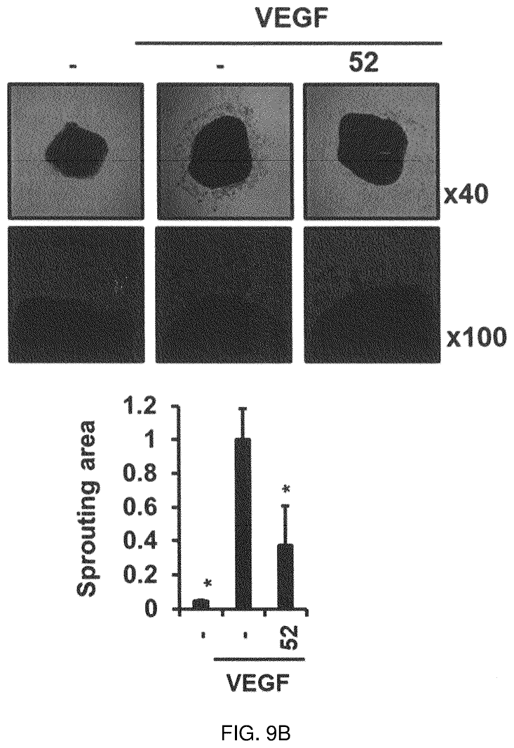

Further, the effect of anti-PTK7 mAbs on ex vivo angiogenesis was evaluated using the mouse aortic ring assay. Treatment with 20 ng/ml VEGF induced sprouting and outgrowth of endothelial cells from the aortas. However, it was confirmed that the inhibition of endothelial cell proliferation in the mAb-32 (10 μg/mL)-treated group and the mAb-43 (10 μg/mL)-treated group was similar to that in the sPTK7 (4 μg/mL)-treated group, and the inhibition of endothelial cell proliferation in the mAb-52 (10 μg/mL)-treated group was also similar to that in the mAb-32- and mAb-43-treated groups (FIG. 9).

(5) Effect of Anti-PTK7 mAbs on Angiogenesis In Vivo

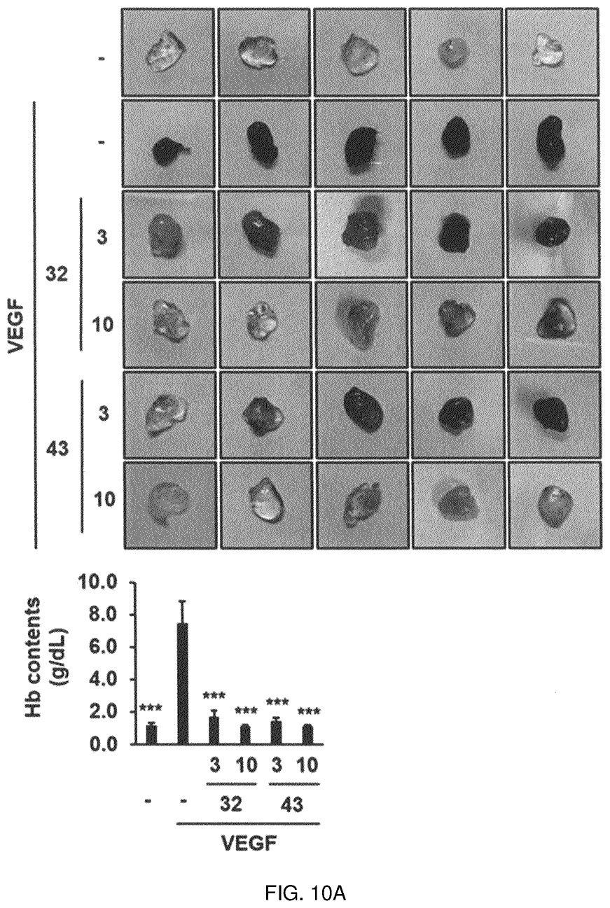

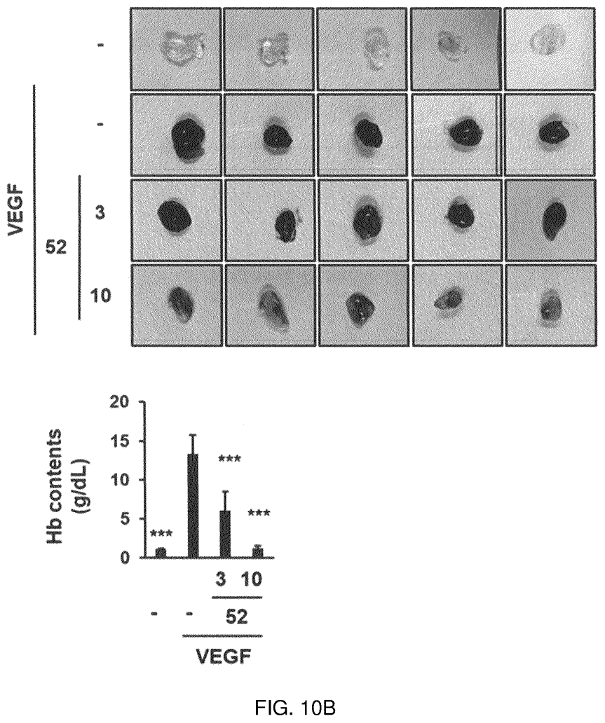

To examine the effect of anti-PTK7 mAbs on angiogenesis in vivo, a Matrigel plug assay was performed. As a result, treatment with mouse VEGF resulted in plugs with a dark red color, indicating induction of angiogenesis. As a result of co-treatment with 3 μg/mL of mAb-32, mAb-43, or mAb-52 and VEGF it was confirmed that plugs with an orange or pale red color were generated, and as a result of co-treatment with 10 μg/mL of mAb-32, mAb-43 or mAb-52 and VEGF, it was confirmed that plugs with a white or yellow color were generated (FIG. 10). Furthermore, the degree of in vivo angiogenesis was quantified by measuring the hemoglobin (Hb) content in the plugs. As a result, the hemoglobin content in the plugs retrieved from mice treated with VEGF was 7.48±1.33 g/dL, but it was confirmed that co-treatment with 3 or 10 μg/ml mAb-32 and VEGF decreased the Hb levels to 1.73±0.36 or 1.15±0.49 g/dL, respectively, and co-treatment with 3 or 10 μg/mL mAb-43 and VEGF decreased the Hb levels to 1.44±0.19 or 1.13±0.06 g/dL, respectively. Furthermore, an independent analysis confirmed that the hemoglobin content in plugs retrieved from mice treated with VEGF was 13.34±2.46 g/dL, whereas co-treatment with 3 or 10 μg/mL mAb-52 and VEGF reduced the hemoglobin levels to 6.08±2.43 or 1.22±0.32 g/dL, respectively (FIG. 10). Therefore, it was confirmed that mAb-32, mAb-43, and mAb-52 concentration-dependently inhibited VEGF-induced angiogenesis in vivo.

(6) Effect of Anti-PTK7 mAbs on VEGF-Induced KDR Signaling in HUVECs

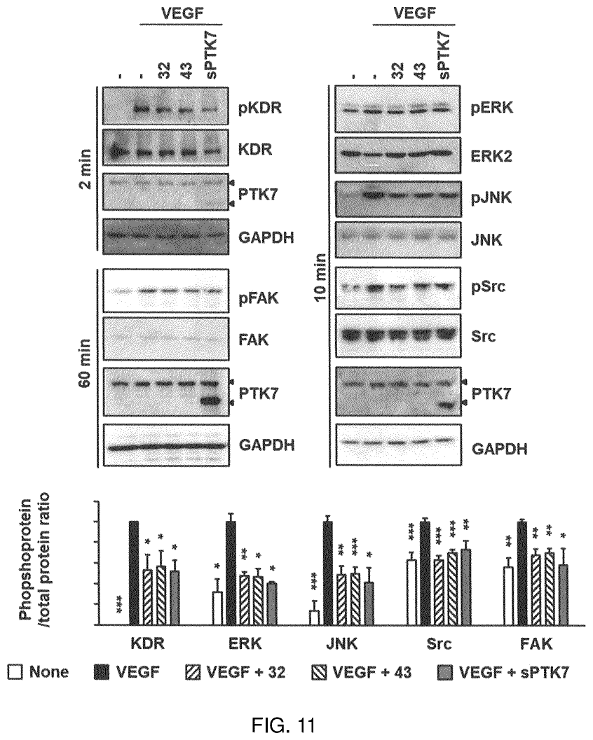

Angiogenesis is mediated by various signaling pathways, including ERK and JNK signaling pathways involved in cell proliferation and differentiation and FAK and Src signaling pathways involved in cell adhesion and migration. Therefore, the effect of anti-PTK7 mAbs on the VEGF-induced activation of signaling proteins in HUVECs was examined. As a result, it was confirmed that mAb-32 and mAb-43 (10 μg/mL each) downregulated the phosphorylation of KDR, ERK, JNK, FAK, and Src (FIG. 11). These results indicate that anti-PTK7 mAbs downregulate the activation of VEGF-induced KDR and downstream signaling pathways involved in angiogenesis.

(7) Effect of Anti-PTK7 mAbs on PTK7-KDR Interaction

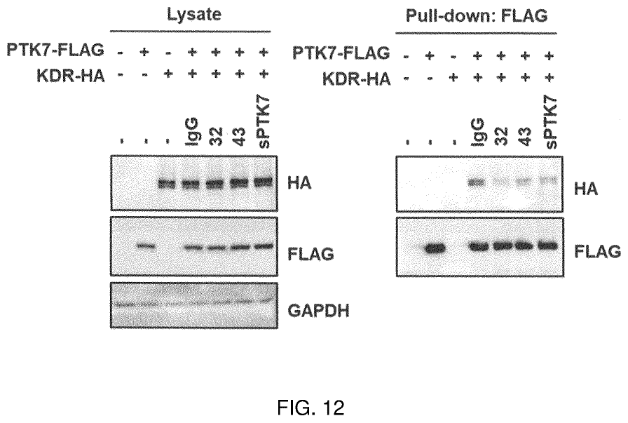

The effect of anti-PTK7 mAbs on PTK7-KDR interaction was examined. PTK7-KDR interaction in HEK293 cells expressing PTK7 and KDR was analyzed by co-precipitation of KDR with PTK7-His-pulled-down proteins with Ni²+-NTA resin after treatment with mAb-32 or mAb-43. As a result, compared with bovine IgG (10 μg/mL), sPTK7 inhibited the binding of PTK7 to KDR. Therefore, it was confirmed that sPTK7 (4 μg/mL) decreased the PTK7-KDR interaction by competing with PTK. Under these conditions, mAb-32 and mAb-43 (10 μg/mL) decreased the binding of PTK7 to KDR (FIG. 12). These results indicate that anti-PTK7 mAbs downregulate VEGF-induced KDR activation and its downstream signaling pathways by inhibiting PTK7-KDR interaction.

(8) Effect of Anti-PTK7 mAbs in Mouse Models Xenografted with TNBC and ESCC Cells

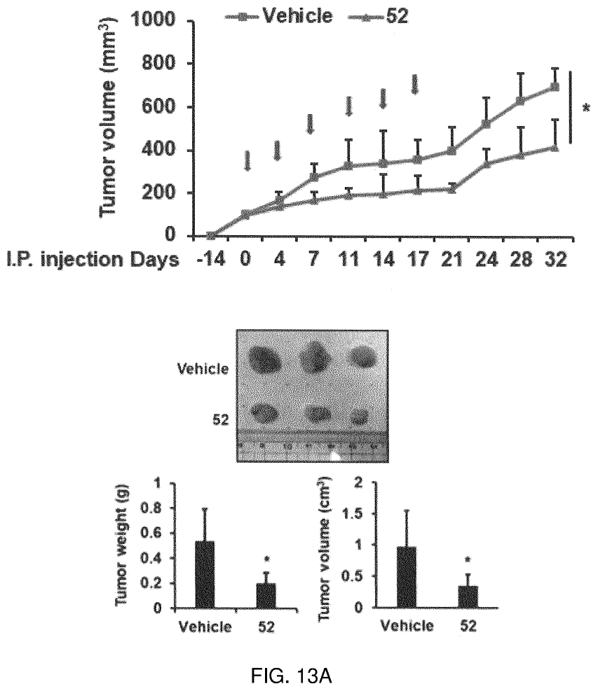

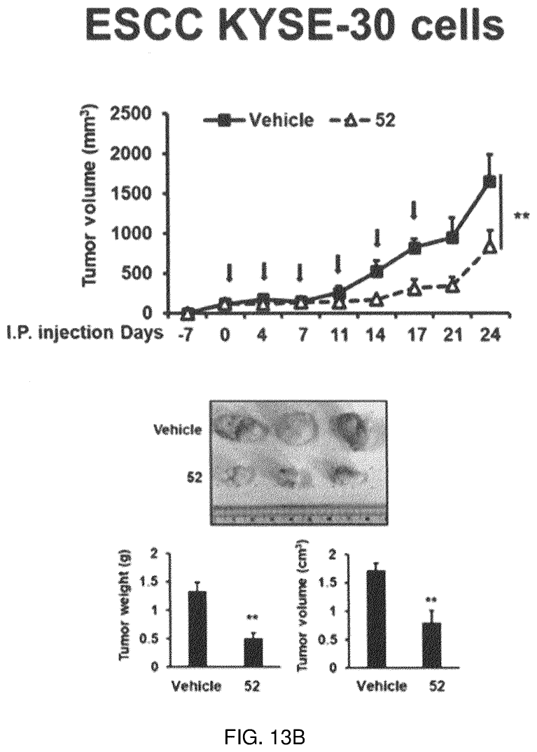

To evaluate the in vivo anti-cancer effects of anti-PTK7 mAbs, the anti-cancer efficacy of anti-PTK7 mAb-52 was analyzed in mice xenografted with TNBC MDA-MB-231 cells and ESCC KYSE-30 cells. In the xenograft model of TNBC MDA-MB-231 cells, mAb-52 was administered intraperitoneally at 10 mg/kg six times over a three-week period. After two weeks, tumor size was reduced to 60.1% compared to the control (FIG. 13A). Similarly, in the xenograft model of ESCC KYSE-30 cells, mAb-52 was injected intraperitoneally, and after one week, tumor size was reduced to 50.6% compared to the control [FIG. 13B]. In the xenograft model of TNBC MDA-MB-231 cells, excised tumors from the control group weighed 0.54±0.26 g and measured 0.97±0.58 cm3, whereas tumors from mAb-52-treated group were reduced to 0.20±0.08 g in weight and 0.34±0.19 cm3 in size. Similarly, in the xenograft model of ESCC KYSE-30 cells, excised tumors from the control group weighed 1.33±0.15 g and measured 1.72±0.12 cm3, whereas tumors from mAb-52-treated group were reduced to 0.50±0.098 g in weight and 0.80±0.21 cm3 in size. These findings indicate that anti-PTK7 mAb-52 effectively inhibits tumor growth in vivo.

1. An anti-PTK7 antibody or a functional fragment thereof, which specifically binds to protein tyrosine kinase 7 (PTK7) and comprises a heavy chain variable region and a light chain variable region,
wherein the heavy chain variable region comprises CDR1-VH comprising an amino acid sequence of SEQ ID NO: 1, 6, 11, or 16, CDR2-VH comprising an amino acid sequence of SEQ ID NO: 2, 7, 12, or 17, and CDR3-VH comprising an amino acid sequence of SEQ ID NO: 3, 8, 13, or 18, and
the light chain variable region comprises CDR1-VL comprising an amino acid sequence of SEQ ID NO: 4, 9, 14, or 19, CDR2-VL comprising Trp-Ala-Ser (WAS) or Ala-Ala-Ser (AAS), and CDR3-VL comprising an amino acid sequence of SEQ ID NO: 5, 10, 15, or 20.

2. The anti-PTK7 antibody or the functional fragment thereof of claim 1, wherein the antibody or the functional fragment thereof comprises a heavy chain variable region composed of an amino acid sequence of SEQ ID NO: 21 and a light chain variable region composed of an amino acid sequence of SEQ ID NO: 22.

3. The anti-PTK7 antibody or the functional fragment thereof of claim 1, wherein the antibody or the functional fragment thereof specifically binds to an extracellular region of a PTK7 protein.

4. The anti-PTK7 antibody or the functional fragment thereof of claim 1, wherein the antibody is one or more selected from the group consisting of lgG, IgA, IgM, IgE, and IgD, and the functional fragment is one or more selected from the group consisting of diabody, Fab, F(ab′), F(ab′)2, Fv, dsFv, and scFv.

5. The anti-PTK7 antibody or the functional fragment thereof of claim 1, wherein the antibody or the functional fragment thereof inhibits one or more selected from the group consisting of adhesion, wound healing, chemotactic migration, and invasion, and angiogenesis.

6. The anti-PTK7 antibody or the functional fragment thereof of claim 1, wherein the antibody or the functional fragment thereof reduces hemoglobin (Hb) levels in a tissue.

7. The anti-PTK7 antibody or the functional fragment thereof of claim 1, wherein the antibody or the functional fragment thereof inhibits the phosphorylation of one or more signaling molecules selected from the group consisting of kinase insert domain receptor (KDR), extracellular-signal-regulated kinase (ERK), c-Jun N-terminal kinase (JNK), focal adhesion kinase (FAK), and tyrosine kinase Src (Src).

8. The anti-PTK7 antibody or the functional fragment thereof of claim 1, wherein the antibody or the functional fragment thereof inhibits the interaction between protein tyrosine kinase 7 (PTK7) and kinase insert domain receptor (KDR).

9. The anti-PTK7 antibody or the functional fragment thereof of claim 1, wherein the antibody or the functional fragment thereof inhibits cancer growth.

10.-14. (canceled)

15. A method of preventing or treating an angiogenesis-associated disease, comprising:
administering the anti-PTK7 antibody or the functional fragment thereof of claim 1 to an individual in need thereof.

16. The method of claim 15, wherein the angiogenesis-associated disease is one or more selected from the group consisting of cancer, endometriosis, obesity, arthritis, arteriosclerosis, hemangioma, angiofibroma, vascular malformation, vascular adhesion, scleroderma, diabetic retinopathy, macular degeneration, neovascular glaucoma, corneal diseases caused by angiogenesis, psoriasis, telangiectasia, pyogenic granuloma, seborrheic dermatitis, and Alzheimer's disease.

17. (canceled)

18. A method of preventing or treating cancer, comprising:
administering the anti-PTK7 antibody or the functional fragment thereof of claim 1 to an individual in need thereof.

19. The method of claim 18, wherein the cancer is one or more selected from the group consisting of glioblastoma, brain cancer, head and neck cancer, breast cancer, lung cancer, esophageal cancer, gastric cancer, duodenal cancer, appendiceal cancer, colorectal cancer, rectal cancer, liver cancer, pancreatic cancer, gallbladder cancer, anal cancer, kidney cancer, ureteral cancer, bladder cancer, prostate cancer, penile cancer, testicular cancer, uterine cancer, ovarian cancer, vulvar cancer, vaginal cancer, and skin cancer.

20. (canceled)


SEQUENCE LISTING (40 sequences)

SEQ ID NO: 1 - protein, 8 aa, synthetic construct
GFDFSRYW

SEQ ID NO: 2 - protein, 8 aa, synthetic construct
INPDSSTI

SEQ ID NO: 3 - protein, 11 aa, synthetic construct
ARAYYIYYFDY

SEQ ID NO: 4 - protein, 12 aa, synthetic construct
QSLLYSSNQKNY

SEQ ID NO: 5 - protein, 9 aa, synthetic construct
QQYYSYPWT

SEQ ID NO: 6 - protein, 8 aa, synthetic construct
GFNIKDTY

SEQ ID NO: 7 - protein, 8 aa, synthetic construct
IDPANGNT

SEQ ID NO: 8 - protein, 10 aa, synthetic construct
ARGDANYGAY

SEQ ID NO: 9 - protein, 10 aa, synthetic construct
ESVDNYGISF

SEQ ID NO: 10 - protein, 9 aa, synthetic construct
QQSKEVPLT

SEQ ID NO: 11 - protein, 8 aa, synthetic construct
GFDFSRYW

SEQ ID NO: 12 - protein, 8 aa, synthetic construct
INPDSSTI

SEQ ID NO: 13 - protein, 11 aa, synthetic construct
ARMELLWYFDV

SEQ ID NO: 14 - protein, 12 aa, synthetic construct
QSLLYSSNQKNY

SEQ ID NO: 15 - protein, 9 aa, synthetic construct
QQYYSYPWT

SEQ ID NO: 16 - protein, 8 aa, synthetic construct
GFNIEDTY

SEQ ID NO: 17 - protein, 8 aa, synthetic construct
IDPANGND

SEQ ID NO: 18 - protein, 10 aa, synthetic construct
ARGDANYGSY

SEQ ID NO: 19 - protein, 10 aa, synthetic construct
ESVDHFGVSF

SEQ ID NO: 20 - protein, 9 aa, synthetic construct
QQSKEVPLT

SEQ ID NO: 21 - protein, 117 aa, synthetic construct
  source 1..117: note = #52-VH
EVQLQQSGAELVKPGASVKLSCTASGFNIEDTYIHWVKQRPEQGLEWIGRIDPANGNDKYDPKFQGKATITADTSSNTAYLQLSSLTSEDTAVYYCARGDANYGSYWGQGTLVTVSA

SEQ ID NO: 22 - protein, 111 aa, synthetic construct
  source 1..111: note = #52-VK
DIVLTQSPASLAVSLGQRATISCRASESVDHFGVSFMNWFQQKPGQPPKLLIYAASNQRSGVPARFSGSGSGTDFSLNIHPMEEDDTAMYFCQQSKEVPLTFGAGTKLELK

SEQ ID NO: 23 - DNA, 352 bp, synthetic construct
  source 1..352: mol_type = other DNA; note = DNA of #52-VK
gaggttcagctgcagcagtctggggcagagcttgtgaagccaggggcctcagtcaagttgtcctgcacagcttctggcttcaacattgaagacacctatatacactgggtgaagcagaggcctgaacagggcctggagtggattggaaggattgatcctgcgaatggtaatgataaatatgacccgaagttccagggcaaggccactataacagcagacacttcctccaacacagcctacctgcagctcagcagcctgacatctgaggacactgccgtctattactgtgctagaggggatgctaactacggttcttactggggccaagggactctggtcactgtctctgcag

SEQ ID NO: 24 - DNA, 333 bp, synthetic construct
  source 1..333: mol_type = other DNA; note = DNA of #52-VH
gacattgtgctgacccaatctccagcttctttggctgtgtctctagggcagagggccaccatctcctgcagagccagcgaaagtgttgatcattttggcgttagttttatgaactggttccagcagaaaccaggacagccacccaaactcctcatctatgctgcatccaaccaaagatccggggtccctgccaggtttagtggcagtgggtctgggacagacttcagcctcaacatccatcctatggaggaggatgatactgcaatgtatttctgtcagcaaagtaaggaggttccgctcacgttcggtgctgggaccaagctggagctgaaa

SEQ ID NO: 25 - DNA, 20 bp, synthetic construct
  source 1..20: mol_type = other DNA; note = Ig1-F
taatacgactcactataggg

SEQ ID NO: 26 - DNA, 47 bp, synthetic construct
  source 1..47: mol_type = other DNA; note = Ig5-His-R
gctctagatcaatgatgatgatgatgatggcctgtggctgaacaggg

SEQ ID NO: 27 - DNA, 47 bp, synthetic construct
  source 1..47: mol_type = other DNA; note = Ig4.2-His-R
gctctagatcaatgatgatgatgatgatgctgggtcaggcaatccaa

SEQ ID NO: 28 - DNA, 47 bp, synthetic construct
  source 1..47: mol_type = other DNA; note = Ig3-His-R
gctctagatcaatgatgatgatgatgatgaaggcaggtcacacgctc

SEQ ID NO: 29 - DNA, 45 bp, synthetic construct
  source 1..45: mol_type = other DNA; note = Ig1-4-His-F
catcactgtggcccatcatcatcatcatcattgatctagagggcc

SEQ ID NO: 30 - DNA, 41 bp, synthetic construct
  source 1..41: mol_type = other DNA; note = Ig1-4-His-R
gatgatgatgatgggccacagtgatgttgacatcctgtctc

SEQ ID NO: 31 - DNA, 40 bp, synthetic construct
  source 1..40: mol_type = other DNA; note = Ig2-4-His-F
gtcttcatcaagcagtggattgaggcaggtcctgtggtcc

SEQ ID NO: 32 - DNA, 42 bp, synthetic construct
  source 1..42: mol_type = other DNA; note = Ig2-4-His-R
cctgcctcaatccactgcttgatgaagacaatggctgtctgg

SEQ ID NO: 33 - DNA, 40 bp, synthetic construct
  source 1..40: mol_type = other DNA; note = Ig3-4-His-F
gtcttcatcaagcaggatgaaagctttgccagggtggtgc

SEQ ID NO: 34 - DNA, 43 bp, synthetic construct
  source 1..43: mol_type = other DNA; note = Ig3-4-His-R
ggcaaagctttcatcctgcttgatgaagacaatggctgtctgg

SEQ ID NO: 35 - protein, 118 aa, synthetic construct
  source 1..118: note = #32-VH
EVKLLESGGGLVQPGGSLKLSCAASGFDFSRYWMNWVRQAPGKGLEWIGEINPDSSTINYTPSLKDKFIISRDNAKNTLYLQMSKVRSEDTALYYCARAYYIYYFDYWGQGTTLTVSS

SEQ ID NO: 36 - protein, 117 aa, synthetic construct
  source 1..117: note = #43-VH
EVLLQQSGADLVKPGASVKLSCTASGFNIKDTYIHWVKQRPEQGLEWIGRIDPANGNTKYDPKFQGKATITADTSSNTAYLQFSSLTSEDTAVYYCARGDANYGAYWGQGTLVTVSA

SEQ ID NO: 37 - protein, 118 aa, synthetic construct
  source 1..118: note = #50-VH
EVNLLESGGGLVQPGGSLKLSCAASGFDFSRYWLSWVRQAPGKGLEWIGEINPDSSTINYTPSLKDKFIISRDNAKNTLYLQMSKVRSEDTALYYCARMELLWYFDVWGAGTTVTVSS

SEQ ID NO: 38 - protein, 113 aa, synthetic construct
  source 1..113: note = #32-VK
DIVWSQSPSSLAVSVGEKVTMSCKSSQSLLYSSNQKNYLAWYQQKPGQSPKLLIYWASIRESGVPDRFTGSGSGTDFTLTISSVKAEDLAVYYCQQYYSYPWTFGGGTKLEIK

SEQ ID NO: 39 - protein, 111 aa, synthetic construct
  source 1..111: note = #43-VK
DIVLTQSPASLAVSLGQRATISCRASESVDNYGISFMNWFQQKPGQPPKLLIYAASNGRSGVPARFSGSGSGTDFSLNIHPMEEDDTAMYFCQQSKEVPLTFGAGTKLELK

SEQ ID NO: 40 - protein, 113 aa, synthetic construct
  source 1..113: note = #50-VK
DIVMSQSPSSLAVSVGEKVTMSCKSSQSLLYSSNQKNYLAWYQQKPGQSPKLLIYWASTRESGVPDRFTGSGSGTDFTLTISSVKAEDLAVYYCQQYYSYPWTFGGGTKLEIK